(19) United States
(12) Patent Application Publication
(10) Pub. No.: US 2010/0280003 A1
(43) Pub. Date: Nov. 4, 2010

(54) COMBINATIONS OF JAK INHIBITORS

(76) Inventors: Nigel Graham Cooke, Oberwil (CH); Paul W. Manley, Arlesheim (CH)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/834,309

(22) Filed: Jul. 12, 2010

Related U.S. Application Data
(63) Continuation of application No. 11/719,838, filed on May 21, 2007, now abandoned.
(60) Provisional application No. 60/630,713, filed on Nov. 24, 2004.

(51) Int. Cl. A61K 31/553; A61K 31/5377; A61K 31/4025; A61K 31/517; A61K 31/47; A61P 35/00

(52) U.S. Cl. 514/211.08; 514/236.2; 514/422; 514/306; 514/307

(57) ABSTRACT

The invention provides a pharmaceutical combination comprising:
a) at least one agent selected from Bcr-Abl, Flt-3, FAK and RAF kinase inhibitors; and
b) at least one JAK kinase inhibitor
   and a method for treating or preventing a proliferative disease using such a combination.

The present invention relates to a pharmaceutical combination comprising at least one subtype selective or subtype non-selective JAK kinase inhibitor and at least one agent selected from Bcr-Abl, Flt-3, RAF and FAK kinase inhibitors, and the uses of such a combination, e.g., in proliferative diseases, e.g., tumors, myelomas, leukemias, psoriasis, restenosis, sclerodermitis and fibrosis.

In spite of numerous treatment options for proliferative disease patients, there remains a need for effective and safe antiproliferative agents and a need for their preferential use in combination therapy.

### SUMMARY OF THE INVENTION

It has now been found that a combination comprising at least one at least one JAK kinase inhibitor, targeting one or more of JAK1, JAK2, JAK3 or TYK2, and at least one agent selected from Bcr-Abl, Flt-3, RAF and FAK kinase inhibitors, e.g., as defined below, has a beneficial effect on proliferative diseases, e.g., tumors, myelomas, leukemias, psoriasis, restenosis, sclerodermitis and fibrosis.

### DETAILED DESCRIPTION OF THE INVENTION

Bcr-Abl is a fusion gene which encodes a 210-kd protein with deregulated tyrosine kinase activity and is present in the leukemia cells of almost every patient with chronic myeloid leukemia (CML) and approximately 33% of patients with acute lymphoblastic leukemia (ALL). Bcr-Abl inhibitors are, e.g., compounds having an IC₅₀ value <5 μM, preferably <1 μM, more preferably <0.1 μM in the following assays:

Test for activity against Bcr-Abl: The murine myeloid progenitor cell line 32Dcl3 transfected with the p210 Bcr-Abl expression vector pGDp210Bcr/Abl (32D-Bcr/Abl) was obtained from J. Griffin (Dana Farber Cancer Institute, Boston, Mass., USA). The cells express the fusion Bcr-Abl protein with a constitutively active Abl kinase and proliferate growth factor independent. The cells are expanded in RPMI 1640 (AMIMED), 10% fetal calf serum, 2 mM glutamine (Gibco) (“complete medium”) and a working stock is prepared by freezing aliquots of 2×10⁶ cells per vial in freezing medium (95% FCS, 5% DMSO (SIGMA)). After thawing, the cells are used during maximally 10-12 passages for the experiments.

For cellular assays, compounds are dissolved in DMSO and diluted with complete medium to yield a starting concentration of 10 μM followed by preparation of serial 3-fold dilutions in complete medium. 200,000 32D-Bcr/Abl cells in 50 μL complete medium are seeded per well in 96-well, round-bottom tissue culture plates. Fifty (50) μL per well of serial 3-fold dilutions of the test compound are added to the cells in triplicates. Untreated cells are used as control. The compound is incubated together with the cells for 90 min. at 37° C., 5% CO₂, followed by centrifugation of the tissue culture plates at 1,300 rpm (Beckmann GPR centrifuge) and removal of the supernatants by careful aspiration taking care not to remove any of the pelleted cells. The cell pellets are lysed by addition of 150 μL lysis buffer (50 mM Tris/HCl, pH 7.4, 150 mM sodium chloride, 5 mM EDTA, 1 mM EGTA, 1% NP-40, 2 mM sodium ortho-vanadate, 1 mM PMSF, 50 μg/mL aprotinin and 80 μg/mL leupeptin) and either used immediately for the ELISA or stored frozen in the plates at −20° C. until usage.

Black ELISA plates (Packard HTRF-96 black plates) are precoated over night at 4° C. with 50 ng/well of the rabbit polyclonal anti-abl-SH3 domain Ab 06-466 from Upstate in 50 μL PBS. After washing 3 times with 200 μL/well PBS containing 0.05% Tween20 (PBST) and 0.5% TopBlock (Juro), residual protein binding sites are blocked with 200 μL/well PBST, 3% TopBlock for 4 hours at room temperature followed by incubation with 50 μL lysates of untreated or compound-treated cells (20 μg total protein per well) for 3-4 hours at 4° C. After 3 washings, 50 μL/well anti-phosphotyrosine Ab PY20(AP) labeled with alkaline phosphatase (Zymed) diluted to 0.2 μg/mL in blocking buffer is added and incubated overnight (4° C.). For all incubation steps the plates are covered with plate sealers (Costar). Finally, the plates are washed another three times with washing buffer and once with deionized water before the addition of 90 μL/well of the AP-substrate CDPStar RTU with Emerald II. After being sealed with Packard TopSeal™-A plate sealers, the plates are incubated for 45 min. at room temperature in the dark and luminescence is quantified by measuring counts per second (CPS) with a Packard Top Count Microplate Scintillation Counter (Top Count).

The difference between the ELISA-readout (CPS) obtained for with the lysates of the untreated 32D-Bcr/Abl cells and the readout for the assay-background (all components, but without cell lysate) is calculated and taken as 100% reflecting the constitutively phosphorylated Bcr-Abl protein present in these cells. The activity of the compound on the Bcr-Abl kinase activity is expressed as percent reduction of the Bcr-Abl phosphorylation. The values for the IC₅₀ and IC₉₀ are determined from the dose response curves by graphical extrapolation.

Suitable Bcr-Abl inhibitors include e.g.:
Compounds as disclosed in U.S. Pat. No. 5,521,184, e.g., an N-phenyl-2-pyrimidine-amine derivative of formula (I):

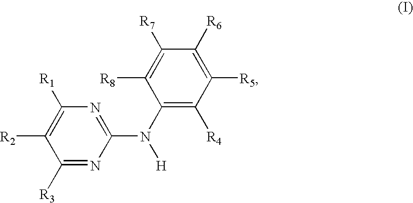

wherein
R₁ is 4-pyrazinyl, 1-methyl-1H-pyrrolyl, amino- or amino-lower alkyl-substituted phenyl wherein the amino group in each case is free, alkylated or acylated, 1H-indolyl or 1H-imidazolyl bonded at a 5-membered ring carbon atom, or unsubstituted or lower alkyl-substituted pyridyl bonded at a ring carbon atom and unsubstituted or substituted at the nitrogen atom by oxygen;
R₂ and R₃ are each independently of the other hydrogen or lower alkyl,
one or two of the radicals R₄, R₅, R₆, R₇ and R₈ are each nitro, fluoro-substituted lower alkoxy or a radical of the formula

—N(R₉)—C(═X)—(Y)ₙ—R₁₀,
wherein
   R₉ is hydrogen or lower alkyl;
   X is oxo, thio, imino, N-lower alkyl-imino, hydroximino or O-lower alkyl-hydroximino;
   Y is oxygen or the group NH;
   n is 0 or 1; and
   R₁₀ is an aliphatic radical having at least 5 carbon atoms, or an aromatic, aromatic-aliphatic, cycloaliphatic, cycloaliphatic-aliphatic, heterocyclic or heterocyclic-aliphatic radical;
and the remaining radicals R₄, R₅, R₆, R₇ and R₈ are each independently of the others hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterified hydroxy, free, alkylated or acylated amino or free or esterified carboxy;
   or a salt of such a compound having at least one salt-forming group.

Examples of compounds of formula (I) include:
N-(3-nitro-phenyl)-4-(3-pyridyl)-2-pyrimidine-amine;
N-[3-(4-chlorobenzoylamido)-phenyl]-4-(3-pyridyl)-2-pyrimidine-amine;
N-(3-benzoylamido-phenyl)-4-(3-pyridyl)-2-pyrimidine-amine;
N-[3-(2-pyridyl)carboxamido-phenyl]-4-(3-pyridyl)-2-pyrimidine-amine;
N-[3-(3-pyridyl)carboxamido-phenyl]-4-(3-pyridyl)-2-pyrimidine-amine;
N-[3-(4-pyridyl)carboxamido-phenyl]-4-(3-pyridyl)-2-pyrimidine-amine;
N-(3-pentafluoro-benzoylamido-phenyl)-4-(3-pyridyl)-2-pyrimidine-amine;
N-[3-(2-carboxy-benzoylamido)-phenyl]-4-(3-pyridyl)-2-pyrimidine-amine;
N-(3-n-hexanoylamido-phenyl)-4-(3-pyridyl)-2-pyrimidine-amine;
N-(3-nitro-phenyl)-4-(2-pyridyl)-2-pyrimidine-amine;
N-(3-nitro-phenyl)-4-(4-pyridyl)-2-pyrimidine-amine;
N-[3-(2-methoxy-benzoylamido)-phenyl]-4-(3-pyridyl)-2-pyrimidine-amine;
N-[3-(4-fluoro-benzoylamido)-phenyl]-4-(3-pyridyl)-2-pyrimidine-amine;
N-[3-(4-cyano-benzoylamido)-phenyl]-4-(3-pyridyl)-2-pyrimidine-amine;
N-[3-(2-thienylcarboxamido)-phenyl]-4-(3-pyridyl)-2-pyrimidine-amine;
N-(3-cyclohexylcarboxamido-phenyl)-4-(3-pyridyl)-2-pyrimidine-amine;
N-[3-(4-methyl-benzoylamido)-phenyl]-4-(3-pyridyl)-2-pyrimidine-amine;
N-[3-(4-chloro-benzoylamido)-phenyl]-4-(4-pyridyl)-2-pyrimidine-amine;
N-{3-[4-(4-methyl-piperazinomethyl)-benzoylamido]-phenyl}-4-(3-pyridyl)-2-pyrimidine-amine;
N-(5-benzoylamido-2-methyl-phenyl)-4-(3-pyridyl)-2-pyrimidine-amine;
N-{5-[4-(4-methyl-piperazino-methyl)-benzoylamido]-2-methyl-phenyl}-4-(3-pyridyl)-2-pyrimidine-amine;
N-[5-(4-methyl-benzoylamido)-2-methyl-phenyl]-4-(3-pyridyl)-2-pyrimidine-amine;
N-[5-(2-naphthoylamido)-2-methyl-phenyl]-4-(3-pyridyl)-2-pyrimidine-amine;
N-[5-(4-chloro-benzoylamido)-2-methyl-phenyl]-4-(3-pyridyl)-2-pyrimidine-amine;
N-[5-(2-methoxy-benzoylamido)-2-methyl-phenyl]-4-(3-pyridyl)-2-pyrimidine-amine;
N-(3-trifluoromethoxy-phenyl)-4-(3-pyridyl)-2-pyrimidine-amine;
N-(3-[1,1,2,2-tetrafluoro-ethoxy]-phenyl)-4-(3-pyridyl)-2-pyrimidine-amine;
N-(3-nitro-5-methyl-phenyl)-4-(3-pyridyl)-2-pyrimidine-amine;
N-(3-nitro-5-trifluoromethyl-phenyl)-4-(3-pyridyl)-2-pyrimidine-amine;
N-(3-nitro-phenyl)-4-(N-oxido-3-pyridyl)-2-pyrimidine-amine;
N-(3-benzoylamido-5-methyl-phenyl)-4-(N-oxido-3-pyridyl)-2-pyrimidine-amine;
   or pharmaceutically acceptable salts thereof.
   Additional Bcr-Abl inhibitor compounds include those disclosed in WO 04/005281, e.g., a compound of formula (II):

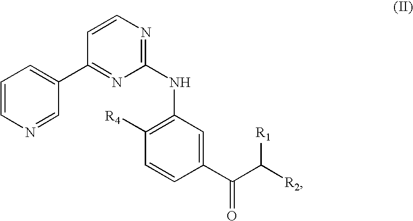

wherein
R₁ represents hydrogen, lower alkyl, lower alkoxy-lower alkyl, acyloxy-lower alkyl, carboxy-lower alkyl, lower alkoxycarbonyl-lower alkyl or phenyl-lower alkyl;
R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;
R₃ represents hydroxy, lower alkoxy, acyloxy, carboxy, lower alkoxycarbonyl, carbamoyl, N-mono- or N,N-di-substituted carbamoyl, amino, mono- or di-substituted amino, cycloalkyl, heterocyclyl, an aryl group, or a mono- or bi-cyclic heteroaryl group comprising zero, one, two or three ring nitrogen atoms and zero or one oxygen atom and zero or one sulfur atom, which groups in each case are unsubstituted or mono- or poly-substituted, or wherein
R₁ and R₂ together represent alkylene with four, five or six carbon atoms optionally mono- or di-substituted by lower alkyl, cycloalkyl, heterocyclyl, phenyl, hydroxy, lower alkoxy, amino, mono- or di-substituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-di-substituted carbamoyl-lower alkyl, cycloalkyl, lower alkoxycarbonyl, carboxy, phenyl, substituted phenyl, pyridinyl, pyrimidinyl or pyrazinyl; and
R₄ represents hydrogen, lower alkyl or halogen;
   and a N-oxide or a pharmaceutically acceptable salt of such a compound.

Examples of compound according to formula (II) include:
4-methyl-3-[[4-(3-pyridinyl)-2-pyrimidinyl]amino]-N-[5-(4-morpholinyl)-3-(trifluoromethyl)phenyl]benzamide;
4-methyl-3-[[4-(3-pyridinyl)-2-pyrimidinyl]amino]-N-[5-(2-methyl-1H-imidazol-1-yl)-3-(trifluoromethyl)phenyl]benzamide;
4-methyl-3-[[4-(3-pyridinyl)-2-pyrimidinyl]amino]-N-[5-(4-methyl-1H-imidazol-1-yl)-3-(trifluoromethyl)phenyl]benzamide;
4-methyl-3-[[4-(3-pyridinyl)-2-pyrimidinyl]amino]-N-[5-(5-methyl-1H-imidazol-1-yl)-3-(trifluoromethyl)phenyl]benzamide;
4-methyl-3-[[4-(3-pyridinyl)-2-pyrimidinyl]amino]-N-[3-(4-methyl-1-piperazinyl)-5-(trifluoromethyl)phenyl]benzamide;
4-methyl-3-[[4-(3-pyridinyl)-2-pyrimidinyl]amino]-N-[2-(1-pyrrolidinyl)-5-(trifluoromethyl)phenyl]benzamide;
   and pharmaceutically acceptable salts thereof.
   Additional Bcr-Abl compounds include those disclosed in EP2005/009967 filed Sep. 16, 2005, namely compounds of the formula (III)

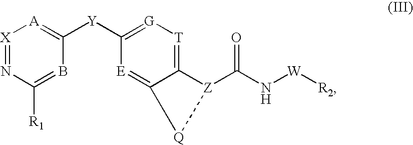

wherein
R₁ is H, halo, —C₀-C₇—O—R₃, —C₀-C₇—NR₄R₅ or —C(═O)—R₆;
R₂ is substituted C₃-C₈-cycloalkyl, substituted aryl or substituted heterocyclyl;
R₃ is H or unsubstituted or substituted lower alkyl;
R₄ and R₅ are independently selected from the group consisting of H, unsubstituted or substituted lower alkyl; lower alkyl-carbonyl, wherein the lower alkyl moiety is optionally substituted and lower alkoxy-carbonyl, wherein the lower alkyl moiety is optionally substituted;
R₆ is H, unsubstituted or substituted lower alkyl, lower alkoxy, wherein the lower alkyl moiety is optionally substituted or unsubstituted, mono- or di-substituted amino;
A, B and X are independently selected from ═C(R₇)— or N;
E, G and T are independently selected from ═C(R₈)— or N;
R₇ and R₈ are independently selected from the group consisting of H, halo and unsubstituted or substituted lower alkyl;
Y is —O—, —S—, —S(O)—, —S(O)₂—, —CH₂— or —CH₂—CH₂—;
Z is CH or N and Q is C₁-C₄-alkylene or C₂-C₄-alkenylene, wherein C₁-C₄-alkylene or C₂-C₄-alkenylene optionally may be substituted and wherein one or more of the carbon atoms of said C₁-C₄-alkylene or C₂-C₄-alkenylene chain optionally may be replaced by a heteroatom independently selected from nitrogen, oxygen and sulfur; and the bond between Q and Z characterized by a dotted line is a single bond; with the proviso that if Z is N, Q is not unsubstituted unbranched C₁-C₄-alkylene, or
Z is C and Q is as defined above, wherein the bond between Q and Z characterized by a dotted line is a double bond; and
W is either not present or C₁-C₃-alkylene;
   or a tautomer thereof, or a salt thereof.

Especially preferred is 6-(6-acetylamino-pyrimidin-4-yloxy)-naphthalene-1-carboxylic acid [4-(4-methyl-piperazin-1-ylmethyl)-3-trifluoromethyl-phenyl]-amide.
Additional Bcr-Abl compound include those disclosed in EP2005/010408 filed Sep. 27, 2005 and U.S. Ser. No. 60/578,491 filed Jun. 10, 2004.

Flt-3 inhibitors are, e.g., compounds having an IC₅₀ value in the range of 1-10,000 nM, preferably in the range of 1-100 nM in the following assays:

Flt-3 kinase inhibition is determined as follows: The baculovirus donor vector pFbacG01 (GIBCO) is used to generate a recombinant baculovirus expressing the amino acid region amino acids 563-993 of the cytoplasmic kinase domain of human Flt-3. The coding sequence for the cytoplasmic domain of Flt-3 is amplified by PCR from human c-DNA libraries (Clontech). The amplified DNA fragments and the pFbacG01 vector are made compatible for ligation by digestion with BamH1 and HindIII. Ligation of these DNA fragments results in the baculovirus donor plasmid Flt-3(1.1). The production of the viruses, the expression of proteins in Sf9 cells and the purification of the GST-fused proteins are performed as follows:

Production of virus: Transfer vector (pFbacG01-Flt-3) containing the Flt-3 kinase domain is transfected into the DH10Bac cell line (GIBCO) and the transfected cells are plated on selective agar plates. Colonies without insertion of the fusion sequence into the viral genome (carried by the bacteria) are blue. Single white colonies are picked and viral DNA (bacmid) is isolated from the bacteria by standard plasmid purification procedures. Sf9 or Sf21 cells (American Type Culture Collection) are then transfected in flasks with the viral DNA using Cellfectin reagent.

Determination of small scale protein expression in Sf9 cells: Virus containing media is collected from the transfected cell culture and used for infection to increase its titre. Virus containing media obtained after two rounds of infection is used for large-scale protein expression. For large-scale protein expression 100 cm² round tissue culture plates are seeded with 5×10⁷ cells/plate and infected with 1 mL of virus-containing media (approximately 5 MOIs). After 3 days, the cells are scraped off the plate and centrifuged at 500 rpm for 5 min. Cell pellets from 10-20, 100 cm² plates, are re-suspended in 50 mL of ice-cold lysis buffer (25 mM Tris-HCl, pH 7.5, 2 mM EDTA, 1% NP-40, 1 mM DTT, 1 mM PMSF). The cells are stirred on ice for 15 min. and then centrifuged at 5,000 rpms for 20 min.

Purification of GST-tagged proteins: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged protein is then eluted by 10 applications (1 mL each) of 25 mM Tris-HCl, pH 7.5, 10 mM reduced-glutathione, 100 mM NaCl, 1 mM DTT, 10% glycerol and stored at −70° C.

Measurement of enzyme activity: Tyrosine protein kinase assays with purified GST-Flt-3 are carried out in a final volume of 30 μL containing 200-1,800 ng of enzyme protein (depending on the specific activity), 20 mM Tris-HCl, pH 7.6, 3 mM MnCl₂, 3 mM MgCl₂, 1 mM DTT, 10 μM Na₃VO₄, 3 μg/mL poly(Glu,Tyr) 4:1, 1% DMSO, 8.0 μM ATP and 0.1 μCi [γ³³P] ATP). The activity is assayed in the presence or absence of inhibitors, by measuring the incorporation of ³³P from [γ³³P] ATP into the poly(Glu,Tyr) substrate. The assay (30 μL) is carried out in 96-well plates at ambient temperature for 20 min. under conditions described below and terminated by the addition of 20 μL of 125 mM EDTA. Subsequently, 40 μL of the reaction mixture is transferred onto Immobilon-PVDF membrane (Millipore, Bedford, Mass., USA) previously soaked for 5 min. with methanol, rinsed with water, then soaked for 5 min. with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of Microscint TM (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein per minute per mg of protein at 37° C. The compounds of the formula (I) here shown IC₅₀ values in the range between 0.005 and 20 μM, preferably between 0.01 and 10 μM.

Suitable Flt-3 inhibitors include, e.g.,
Compounds as disclosed in WO 03/037347, e.g., staurosporine derivatives of formula (IV) or (V):

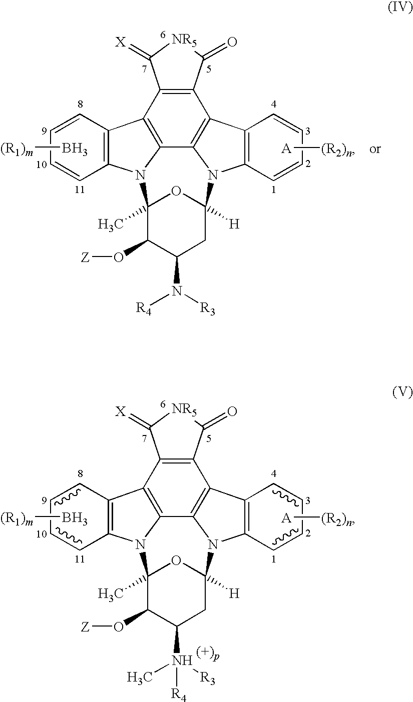

wherein (V) is the partially hydrogenated derivative of compound (IV), or

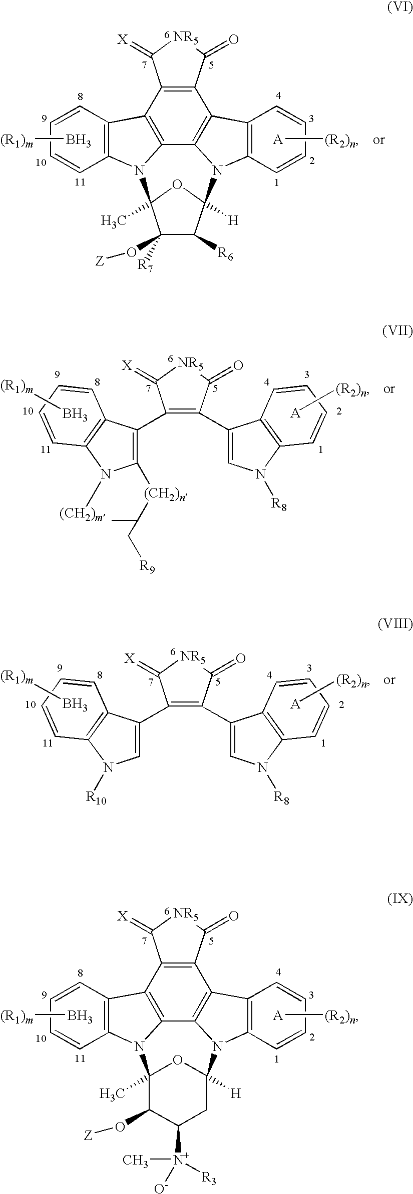

wherein
R₁ and R₂ are, independently of one another, unsubstituted or substituted alkyl, hydrogen, halogen, hydroxy, etherified or esterified hydroxy, amino, mono- or di-substituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 0 to and including 4;
R₃, R₄, R₈ and R₁₀ are, independently of one another, hydrogen, —O⁻, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms, an acyl with up to 30 carbon atoms, wherein R₄ may also be absent, or
if R₃ is acyl with up to 30 carbon atoms, R₄ is not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxy, etherified or esterified hydroxy, amino, mono- or di-substituted amino, cyano, nitro, mercapto, substituted mercapto, carboxy, carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms, for 1 hydrogen atom and hydroxy, for O or for hydrogen and lower alkoxy;
Z stands for hydrogen or lower alkyl;
and either the two bonds characterised by wavy lines are absent in ring A and replaced by 4 hydrogen atoms, and the two wavy lines in ring B each, together with the respective parallel bond, signify a double bond; or
or the two bonds characterised by wavy lines are absent in ring B and replaced by a total of 4 hydrogen atoms, and the two wavy lines in ring A each, together with the respective parallel bond, signify a double bond; or
both in ring A and in ring B all of the 4 wavy bonds are absent and are replaced by a total of 8 hydrogen atoms;
   or a salt thereof, if at least one salt-forming group is present.

Preferably, the FLT-3 inhibitor is N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-Im]pyrrolo[3,4-j][1,7]benzodiazonin-11-yl]-N-methylbenzamide of the formula (X):

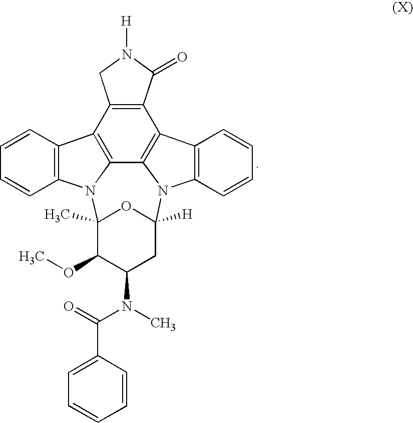

Additional Flt-3 inhibitor compounds include those disclosed in WO 03/099771, e.g., diaryl urea derivatives of the formula (XI):

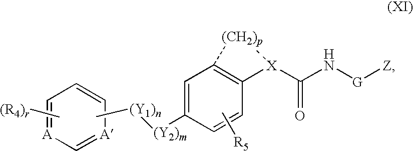

wherein
G is either not present, lower alkylene or C₃-C₅-cycloalkylene; and
Z is a radical of the formula (XIa)

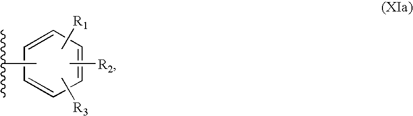

or
G is not present; and
Z is a radical of the formula (XIb)

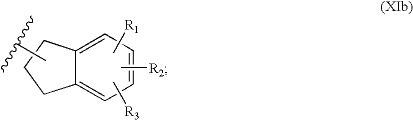

A is CH, N or N→O and A′ is N or N→O, with the proviso that not more than one of A and A′ can be N→O;
n is 1 or 2;
m is 0, 1 or 2;
p is 0, 2 or 3;
r is 0 to 5;
X is NR if p is 0, wherein R is hydrogen or an organic moiety, or if p is 2 or 3, X is nitrogen which together with (CH₂)ₚ and the bonds represented in dotted (interrupted) lines (including the atoms to which they are bound) forms a ring, or
X is CHK, wherein K is lower alkyl or hydrogen and p is zero, with the proviso that the bonds represented in dotted lines, if p is zero, are absent;
Y₁ is O, S or CH₂;
Y₂ is O, S or NH, with the proviso that (Y₁)ₙ—(Y₂)ₘ does not include O—O, S—S, NH—O, NH—S or S—O groups;
each of R₁, R₂, R₃ and R₅, independently of the others, is hydrogen or an inorganic or organic moiety or any two of them together form a lower alkylene-dioxy bridge bound via the oxygen atoms, and the remaining one of these moieties is hydrogen or an inorganic or organic moiety; and
R₄ (if present, that is, if r is not zero) is an inorganic or organic moiety;
   or a tautomer thereof; or a pharmaceutically acceptable salt thereof.

Examples of compounds of formula (XI) include:
N-(4-pyridin-4-yl-oxy-phenyl)-N′-(4-ethyl-phenyl)-urea;
N-(4-pyridin-4-yl-oxy-phenyl)-N′-(3-trifluoromethyl-phenyl)-urea;
N-(4-pyridin-4-yl-oxy-phenyl)-N′-(4-(2,2,2-trifluoroethoxy)-3-trifluoromethyl-phenyl)-urea;
N-(4-(4-(4-hydroxyphenylamino)-pyrimidin-6-yl)-oxyphenyl)-N′-(3-trifluoromethylphenyl)-urea;
N-(4-(2-methyl-pyridin-4-yl)-oxyphenyl)-N′-(3-trifluoromethyl-phenyl)-urea;
N-(4-pyridin-4-yl-oxy-phenyl)-N′-(4-n-propyl-phenyl)-urea;
N-(4-pyridin-4-yl-oxy-phenyl)-N′-(4-methyl-phenyl)-urea;
N-methyl-N-(4-pyridin-4-yl-oxy-phenyl)-N′-(4-ethyl-phenyl)-urea;
N-methyl-N-(4-pyridin-4-yl-oxy-phenyl)-N′-(3-trifluoromethyl-phenyl)-urea;
N-methyl-N-(4-pyridin-4-yl-oxy-phenyl)-N′-(4-n-propyl-phenyl)-urea;
N-methyl-N-(4-pyridin-4-yl-oxy-phenyl)-N′-(4-methyl-phenyl)-urea;
N-(4-pyridin-4-yl-oxy-phenyl)-N′-(4-bromo-3-trifluoromethyl-phenyl)-urea;
N-(4-pyridin-4-yl-oxy-phenyl)-N′-(3-methoxy-5-trifluoromethyl-phenyl)-urea;
N-(4-pyridin-4-ylmethyl-phenyl)-N′-(4-n-propyl-phenyl)-urea;
N-(4-pyridin-4-ylmethyl-phenyl)-N′-(4-ethyl-phenyl)-urea;
N-(4-pyridin-4-ylmethyl-phenyl)-N′-(4-methyl-phenyl)-urea;
N-(4-pyridin-4-ylmethyl-phenyl)-N′-(3-trifluoromethyl-phenyl)-urea;
N-(4-pyridin-4-yl-oxy-phenyl)acetyl-(4-ethyl-phenyl)-amide;
N-(4-pyridin-4-yl-oxy-phenyl)acetyl-(4-methyl-phenyl)-amide;
N-(4-pyridin-4-yl-oxy-phenyl)acetyl-(4-n-propyl-phenyl)amide;
5-(4-pyridyl-oxy)-N-(3-trifluoromethyl-phenyl)amino-carbonyl-2,3-dihydroindole;
5-(4-pyridyl-oxy)-N-(3-trifluoromethyl-phenyl)amino-carbonyl-1,2,3,4-tetrahydroquinoline;
N-(4-(4-chloropyrimidin-6-yl)-oxyphenyl)-N′-(3-trifluoromethylphenyl)-urea;
N-(4-pyridin-4-yl-oxyphenyl)-N′-(4-phenyl-3-trifluoromethyl-phenyl)-urea;
N-(4-pyridin-4-yl-oxyphenyl)-N′-(4-(piperidin-1-yl)-3-trifluoromethyl-phenyl)-urea;
N-(4-pyridin-4-yl-oxyphenyl)-N′-(4-(morpholino)-3-trifluoromethyl-phenyl)-urea;
N-(4-pyridin-4-yl-oxyphenyl)-N′-(3,4,5-trimethoxy-phenyl)-urea;
N-(4-pyridin-4-yl-oxyphenyl)-N′-(3-methoxy-4-phenyl-phenyl)-urea;
N-(4-pyridin-4-yl-oxyphenyl)-N′-(3-methoxy-4,5-(ethylen-1,2-dioxy)-phenyl)-urea;
N-(4-pyridin-4-yl-oxyphenyl)-N′-(3-methoxy-4-(2,2,2-trifluoroethoxy)-phenyl)-urea;
N-(4-pyridin-4-yl-oxyphenyl)-N′-(3-methoxy-4-piperidin-1-yl-phenyl)-urea;
N-(4-pyridin-4-yl-oxyphenyl)-N′-(4-piperidin-1-yl-phenyl)-urea;
N-(4-[2-(4-hydroxyphenyl)-amino-pyrimidin-4-yl]-oxyphenyl-N′-(3-trifluoromethyl-phenyl)-urea;
N-(4-[4-(4-sulfamoylphenyl)-amino-pyrimidin-6-yl]-oxyphenyl-N′-(3-trifluoromethyl-phenyl)-urea;
N-(4-[4-(4-carbamoylphenyl)-amino-pyrimidin-6-yl]-oxyphenyl-N′-(3-trifluoromethyl-phenyl)-urea;
N-(4-[4-(4-(N-2-hydroxyethylcarbamoyl)-phenyl)-amino-pyrimidin-6-yl]-oxyphenyl-N′-(3-trifluoromethyl-phenyl)-urea;
N-(4-[4-(4-hydroxyphenyl)-amino-pyrimidin-6-yl]-oxyphenyl-N′-(3-trifluoromethyl-4-(2,2,2-trifluoroethoxy)-phenyl)-urea;
N-(4-(N-oxido-pyridin-4-yl)-oxyphenyl)-N′-(3-trifluoromethyl-phenyl)-urea;
N-(4-(2-methoxypyridin-5-yl)-oxyphenyl)-N′-(3-trifluoromethyl-phenyl)-urea;
N-(4-(2-pyridon-5-yl)-oxyphenyl)-N′-(3-trifluoromethyl-phenyl)-urea;
N-[4-{(2-acetylamino)-pyridin-4-yl}-oxy]-phenyl-N′-(3-trifluoromethyl-phenyl)-urea;
N-[4-(pyridin-4-yl-oxy)-2-chloro-phenyl]-N′-(3-trifluoromethyl-phenyl)-urea;
N-[4-(pyridin-4-yl-oxy)-2-methyl-phenyl]-N′-(3-trifluoromethyl-phenyl)-urea; and
N-(4-[4-(2-aminoethoxyphenyl)-amino-pyrimidin-6-yl]-oxyphenyl-N′-(3-trifluoromethyl-phenyl)-urea;
   or pharmaceutically acceptable salts thereof.

Most preferred is:
1-[4-(4-ethyl-piperazinyl-1-ylmethyl)-3-trifluoromethyl-phenyl]-3-[4-(6-methylamino-pyrimidin-4-yloxy-phenyl]-urea;
1-[4-(2-amino-pyrimidin-4-yloxy)-phenyl]-3-[4-(4-ethyl-piperazin-1-ylmethyl)-3-trifluoromethyl-phenyl]-urea; and
1-[4-(2-amino-pyrimidin-4-yloxy)-phenyl]-3-[4-(4-methyl-piperazin-1-ylmethyl)-3-trifluoromethyl-phenyl]-urea;
   and pharmaceutically acceptable salts thereof.
   Further Flt-3 inhibitor compounds include those disclosed in WO 04/046120, e.g., compounds of the formula (XII):

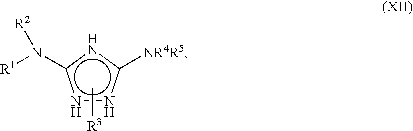

or a pharmaceutically acceptable salt thereof, wherein
R₁ is hydrogen or Y—R′, wherein Y is an optionally substituted C₁-C₆-alkylidene chain wherein up to two methylene units are optionally and independently replaced with —O—, —S—, —NR—, —COO—, —COO— or —CO—; each occurrence of R is independently hydrogen or an optionally substituted C₁-C₆-aliphatic group; and each occurrence of R′ is independently hydrogen or an optionally substituted group selected from a C₁-C₆-aliphatic group, a 3- to 8-membered saturated, partially unsaturated or fully unsaturated monocyclic ring having 0-3 heteroatoms independently selected from nitrogen, oxygen or sulfur, or an 8- to 12-membered saturated, partially unsaturated or fully unsaturated bicyclic ring system having 0-5 heteroatoms independently selected from nitrogen, oxygen or sulfur, or R and R′, two occurrences of R, or two occurrences of R′, are taken together with the atom(s) to which they are bound to form an optionally substituted 3- to 12-membered saturated, partially unsaturated or fully unsaturated monocyclic or bicyclic ring having 0-4 heteroatoms independently selected from nitrogen, oxygen or sulfur;
R² is -(T)ₙAr¹ or -(T)ₙCy¹,
wherein
   T is an optionally substituted C₁-C₄-alkylidene chain wherein one methylene unit of T is optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—;
   n is 0 or 1;
   Ar¹ is an optionally substituted aryl group selected from a 5- to 6-membered monocyclic or an 8- to 12-membered bicyclic ring having 0-5 heteroatoms independently selected from nitrogen, oxygen or sulfur; and
   Cy' is an optionally substituted group selected from a 3- to 7-membered saturated or partially unsaturated monocyclic ring having 0-3 heteroatoms independently selected from nitrogen, oxygen or sulfur, or an 8- to 12-membered saturated or partially unsaturated bicyclic ring system having 0-5 heteroatoms independently selected from nitrogen, oxygen or sulfur, or
R¹ and R², taken together with the nitrogen form an optionally substituted 5- to 8-membered monocyclic or 8- to 12-membered bicyclic saturated, partially unsaturated or fully unsaturated ring having 0-3 additional heteroatoms independently selected from nitrogen, oxygen or sulfur, wherein Ar¹, Cy¹ or any ring formed by R¹ and R² taken together, are each independently optionally substituted with x independent occurrences of Q-R^{X},
wherein
   x is 0-5;
   Q is a bond or is a C₁-C₆-alkylidene chain wherein up to two methylene units of Q are optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—; and
   each occurrence of R^{X} is independently R′, halogen, NO₂, CN, OR′, SR′, N(R′)₂, NR′COR′, NR′CONR′₂, NR′CO₂R′, COR′, CO₂R′, OCOR′, CON(R′)₂, OCON(R′)₂, SOR′, SO₂R′, SO₂N(R′)₂, NR′SO₂R′, NR′SO₂N(R′)₂, COCOR′ or COCH₂COR′;
R³ is bonded to the nitrogen atom in either the 1- or 2-position of the ring and is (L)ₘAr² or (L)ₘCy²,
wherein
   L is an optionally substituted C₁-C₄-alkylidene chain wherein one methylene unit of L is optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—;
   m is 0 or 1;
   Ar² is an optionally substituted aryl group selected from a 5- to 6-membered monocyclic or an 8- to 12-membered bicyclic ring having 0-5 heteroatoms independently selected from nitrogen, oxygen or sulfur; and
   Cy² is an optionally substituted group selected from a 3- to 7-membered saturated or partially unsaturated monocyclic ring having 0-3 heteroatoms independently selected from nitrogen, oxygen or sulfur, or an 8- to 12-membered saturated or partially unsaturated bicyclic ring system having 0-5 heteroatoms independently selected from nitrogen, oxygen or sulfur, wherein Ar² and Cy² are each independently optionally substituted with y occurrences of Z—R^{Y},
   wherein
      y is 0-5;
      Z is a bond or is a C₁-C₆-alkylidene chain wherein up to two methylene units of Z are optionally replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—; and
      each occurrence of R^{Y} is independently R′, halogen, NO₂, CN, OR′, SR′, N(R′)₂, NR′COR′, NR′CONR′₂, NR′CO₂R′, COR′, CO₂R′, OCOR′, CON(R′)₂, OCON(R′)₂, SOR′, SO₂R′, SO₂N(R′)₂, NR′SO₂R′, NR′SO₂N(R′)₂, COCOR′ or COCH₂COR′;
R⁴ is hydrogen or C₁-C₆-alkyl, provided that when R⁵ is hydrogen, R⁴ is also hydrogen;
R⁵ is hydrogen, or
R³ and R⁵, taken together form an optionally substituted group selected from a 5- to 7-membered saturated, partially unsaturated or fully unsaturated monocyclic ring having 0-3 heteroatoms independently selected from nitrogen, oxygen or sulfur, or an 8- to 10-membered saturated, partially unsaturated or fully unsaturated bicyclic ring system having 0-3 heteroatoms independently selected from nitrogen, oxygen or sulfur; and
wherein any ring formed R³ and R⁵ taken together, is optionally substituted with up to five substituents selected from W—R^{W},
wherein
   W is a bond or is a C₁-C₆-alkylidene chain wherein up to two methylene units of W are optionally and independently replaced by —NR—, —S—, —O—, —CS—, —CO₂—, —OCO—, —CO—, —COCO—, —CONR—, —NRCO—, —NRCO₂—, —SO₂NR—, —NRSO₂—, —CONRNR—, —NRCONR—, —OCONR—, —NRNR—, —NRSO₂NR—, —SO—, —SO₂—, —PO—, —PO₂— or —POR—; and
   each occurrence of R^{W} is independently R′, halogen, NO₂, CN, OR′, SR′, N(R′)₂, NR′COR′, NR′CONR′₂, NR′CO₂R′, COR′, CO₂R′, OCOR′, CON(R′)₂, OCON(R)₂, SOR′, SO₂R′, SO₂N(R′)₂, NR′SO₂R′, NR′SO₂N(R′)₂, COCOR′ or COCH₂COR′,
provided that when:
a) R³ is unsubstituted phenyl and R¹ is hydrogen, then R² is not:
   i) unsubstituted phenyl;
   ii) unsubstituted pyridyl;
   iii) benzyl substituted with o-OMe;
   iv) —(C═S)NH(C═O)phenyl;
   v)

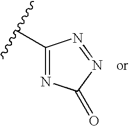

vi) —(C═S)NH-naphthyl or —(C═O)NH-naphthyl, or
b) R³ is substituted or unsubstituted phenyl, then R² is not phenyl substituted in the para position with oxazole, thiazole, thiadiazole, oxadiazole, tetrazole, triazole, diazole or pyrrole;
c) R³ is phenyl, pyridyl, pyrimidinedione or cyclohexyl and R¹ is hydrogen, then R² is not phenyl simultaneously substituted with one occurrence of OMe in the meta position, and one occurrence of oxazole in the para position;
d) R³ is 4-Cl phenyl or 3,4-Cl-phenyl, then R² is not p-Cl phenyl;
e) R³ is unsubstituted pyrimidinyl, then R² is not unsubstituted phenyl, p-OMe substituted phenyl, p-OEt substituted phenyl or o-OMe substituted phenyl or when R³ is 4-Me pyrimidinyl or 4,6-dimethylpyrimidinyl, then R² is not unsubstituted phenyl;
f) compounds of formula (XII) exclude:

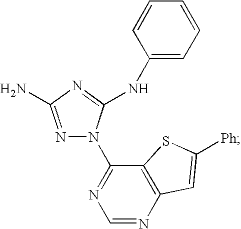

g) R² is 3-pyridinyl and R¹ is hydrogen, then R³ is not trimethoxybenzoyl;
h) R³ is optionally substituted phenyl and R¹ is hydrogen, then R² is not —(C═S)NH(C═O)phenyl, —(C═O)NHphenyl, —(C═S)NHphenyl or —(C═O)CH₂(C═O)-phenyl;
i) R¹ is hydrogen and R² is unsubstituted benzyl, then R³ is not thiadiazole substituted with optionally substituted phenyl;
j) R¹ is hydrogen, R² is pyridyl and R³ is pyridyl, then R² is not substituted with one or more of CF₃, Me, OMe, Br or Cl;
k) R¹ is hydrogen and R² is pyridyl, then R³ is not unsubstituted pyridyl, unsubstituted quinoline, unsubstituted phenyl or unsubstituted isoquinoline;
l) R¹ is hydrogen and R² is unsubstituted quinoline, then R³ is not unsubstituted pyridyl or unsubstituted quinoline;
m) R¹ is hydrogen and R² is unsubstituted isoquinoline or unsubstituted naphthyl, then R³ is not unsubstituted pyridyl;
n) compounds of formula (XII) exclude those compounds having the general structure:

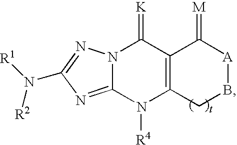

wherein
   R¹, R² and R³ are as defined above; and
   M and K are O or H₂, provided that K and M are different, A and B are each —CH₂—, —NH—, —N-alkyl-, N-aralkyl-, —NCOR^{a}, —NCONHR^{b} or —NCSNHR^{b},
   wherein
      R^{a} is lower alkyl or aralkyl; and
      R^{b} is straight- or branched-chain alkyl, aralkyl or aryl which can either be unsubstituted or substituted with one or more alkyl and/or haloalkyl substituents;
o) compounds of formula (XII) exclude those compounds having the general structure:

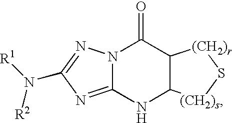

wherein
   R¹ and R² are as defined above; and
   r and s are each independently 0, 1, 2, 3 or 4, provided that the sum of s and r is at least 1;
p) compounds of formula (XII) exclude any one or more of, or all of the following compounds:

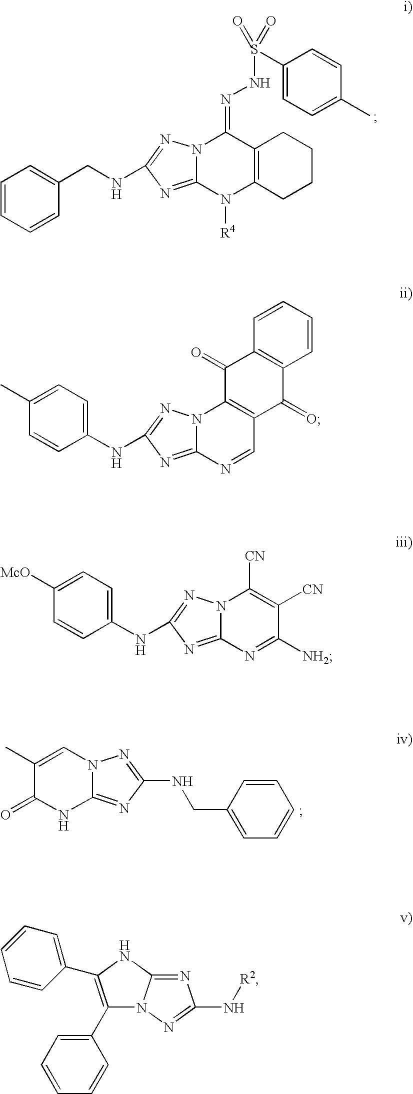

where R² is NH(CH)(Ph)C═O(Ph);

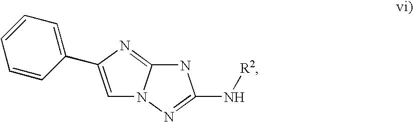

where R² is unsubstituted phenyl or phenyl substituted with OMe, Cl or Me;

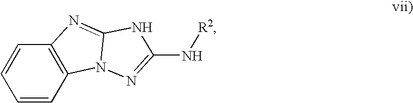

where
   R² is unsubstituted phenyl or phenyl substituted with OMe, Cl, Me or OMe, or
   R² is unsubstituted benzyl;

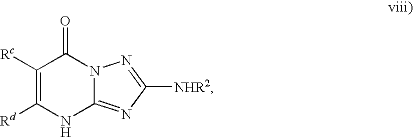

where
   R² is optionally substituted aralkyl; and
   R^{c} and R^{d} are, each independently, Me, hydrogen, CH₂Cl or Cl;

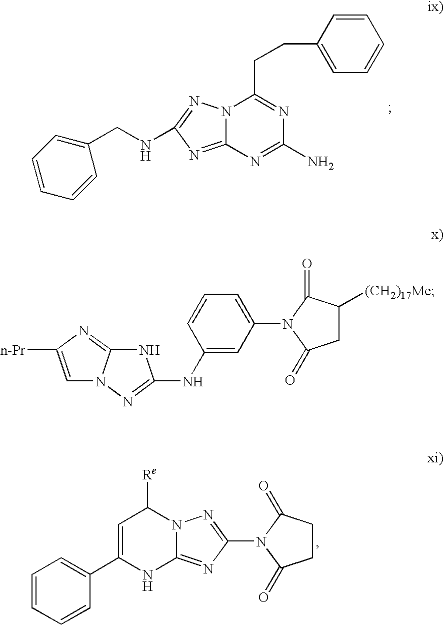

where R^{e} is optionally substituted phenyl;

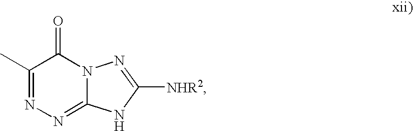

where R² is phenyl optionally substituted with Me, OMe, Br or Cl; or
q) when
   R¹ is hydrogen; and
   R² is phenyl or optionally substituted phenyl; and
   m is 1, then L is not —CO—, —COCH₂— or —COCH═CH—.

Especially preferred is the compound N3-[4-(4-morpholin-4-yl-cyclohexyl)-phenyl]-1-pyridin-2-yl-1H-[1,2,4]triazole-3,5-diamine of the structure:

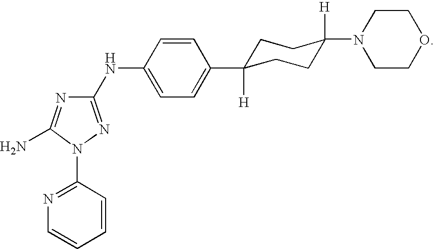

RAF kinase, a serine/threonine kinase that functions in the MAP kinase signaling pathway which is one of the pathways for growth factors to send their signal to proliferate from the extracellular environment to the cell nucleus.

RAF inhibitors are, e.g., compounds which inhibit wild-type C-Raf at an IC₅₀ of from 0.05 mmol/L to more than 4.0 mmol/L and/or mutant B-Raf (V599E) at an IC₅₀ of from 0.08 mmol/L to more than 4.0 mmol/L in the following assays:

Test for activity against the RAF kinase: Active B-Raf, C-Raf and V599E B-Raf proteins of human sequence are purified from insect cells using the baculoviral expression system. Raf inhibition is tested in 96-well microplates coated with IκB-α and blocked with Superblock. The phosphorylation of IκB-α at Serine 36 is detected using a phospho-IκB-α specific antibody (Cell Signaling #9246), an anti-mouse IgG alkaline phosphatase conjugated secondary antibody (Pierce #31320), and an alkaline phosphatase substrate, ATTOPHOS (Promega, #S101).

Suitable RAF inhibitors include, e.g.,
Compounds as disclosed in WO 00/09495, e.g., compounds of formula (XIII):

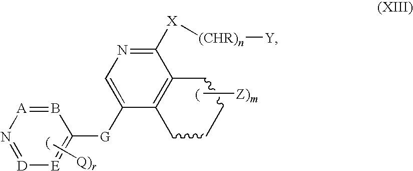

wherein
r is from 0 to 2;
n is from 0 to 2;
m is from 0 to 4;
A, B, D and E are each independently of the others N or CH, with the proviso that not more than two of those radicals are N;
G is lower alkylene, —CH₂—O—, —CH₂—S—, —CH₂—NH—, oxa (—O—), thia (—S—) or imino (—NH—), or is lower alkylene substituted by acyloxy or by hydroxy;
Q is lower alkyl, especially methyl;
R is H or lower alkyl;
X is imino, oxa or thia;
Y is lower alkyl or, especially, aryl, heteroaryl or unsubstituted or substituted cycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m 2), the substituents Z are identical or different;
and wherein the bonds indicated by a wavy line are either single bonds or double bonds;
   or an N-oxide of the mentioned compound, wherein one or more N atoms carry an oxygen atom; or a salt thereof.

Especially preferred is (4-tert-butyl-phenyl)-(4-pyridin-4-ylmethyl-isoquinolin-1-yl)-amine.
Additional RAF inhibitors include compounds disclosed in WO 05/028444, e.g., compounds of formula (XIV):

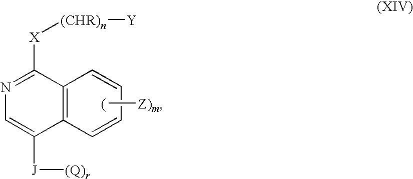

wherein
r is from 0-2;
n is from 0-2;
m is from 0-4;
J is aryl, heteroaryl, cycloalkyl or heterocycloalkyl,
wherein
   aryl is an aromatic radical having from 6-14 carbon atoms, such as phenyl, naphthyl, fluorenyl and phenanthrenyl;
   heteroaryl is an aromatic radical having from 4-14, especially from 5-7 ring atoms, of which 1, 2 or 3 atoms are chosen independently from N, S and O, such as furyl, pyranyl, pyridyl, 1,2-, 1,3- and 1,4-pyrimidinyl, pyrazinyl, triazinyl, triazolyl, oxazolyl, quinazolyl, imidazolyl, pyrrolyl, isoxazolyl isothiazolyl, indolyl, isoindolinyl, quinolyl, isoquinolyl, purinyl, cinnolinyl, naphthyridinyl, phthalazinyl, isobenzofuranyl, chromenyl, purinyl, thianthrenyl, xanthenyl, acridinyl, carbazolyl and phenazinyl;
   cycloalkyl is a saturated cyclic radical having from 3-8, preferably from 5-6 ring atoms, such as cyclopropyl, cyclopentyl and cyclohexyl; and
   heterocycloalkyl is a saturated cyclic radical having from 3-8, preferably from 5-6 ring atoms, of which 1, 2 or 3 atoms are chosen independently from N, S and O, such as piperidyl, piperazinyl, imidazolidinyl, pyrrolidinyl and pyrazolidinyl;
Q is a substituent on 1 or 2 carbon atoms selected from the group consisting of halogen, unsubstituted or substituted lower alkyl, —OR₂, —SR₂, —NR₂, —NRS(O)₂N(R)₂, —NRS(O)₂R, —S(O)R₂, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl, —C₁-C₄-alkyl-aryl, —C₁-C₄-alkyl-heteroaryl, —C₁-C₄-alkyl-heterocyclyl, amino, mono- or di-substituted amino;
R is H or lower alkyl;
R₂ is unsubstituted or substituted alkyl, unsubstituted or substituted cycloalkyl, phenyl, —C₁-C₄-alkyl-aryl, —C₁-C₄-alkyl-heteroaryl or —C₁-C₄-alkyl-heterocycloalkyl;
X is Y, —N(R)—, oxa, thio, sulfone, sulfoxide, sulfonamide, amide or ureylene, preferably —NH—;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
   or an N-oxide of the mentioned compound, wherein one or more N atoms carry an oxygen atom; or a pharmaceutically acceptable salt thereof.

Most preferred are compounds selected from selected from:
[4,7′]biisoquinolinyl-1-yl-4-(tert-butyl-phenyl)-amine;
(4-tert-butyl-phenyl)-(4-quinazolin-6-yl-isoquinolin-1-yl)-amine; and
[4,7′]biisoquinolinyl-1-yl-(2-tert-butyl-pyrimidin-5-yl)-amine.

Focal Adhesion Kinase (FAK) is a key enzyme in the integrin-mediated outside-in signal cascade (D. Schlaepfer et al., Prog Biophys Mol Biol, Vol. 71, pp. 435-478 (1999). Interaction between cells and extracellular matrix (ECM) proteins is transduced as intracellular signals important for growth, survival and migration through cell surface receptors, integrins. FAK plays an essential role in these integrin-mediated outside-in signal cascades. The trigger in the signal transduction cascade is the autophosphorylation of Y397. Phosphorylated Y397 is a SH2 docking site for Src family tyrosine kinases. The bound c-Src kinase phosphorylates other tyrosine residues in FAK. Among them, phsophorylated Y925 becomes a binding site for the SH2 site of Grb2 small adaptor protein. This direct binding of Grb2 to FAK is one of the key steps for the activation of down stream targets, such as the Ras-ERK2/MAP kinase cascade.

Compounds of the invention are active in a FAK assay system as described in the Examples, and show an inhibition IC₅₀ in the range of 1-100 nM. Particularly active are the compounds show IC₅₀ vales in the range of 1-5 nM.

FAK inhibition is determined as follows: All steps are performed in a 96-well black microtiter plate. Purified recombinant hexahistidine-tagged human FAK kinase domain is diluted with dilution buffer (50 mM HEPES, pH 7.5, 0.01% BSA, 0.05% Tween-20 in water) to a concentration of 94 ng/mL (2.5 nM). The reaction mixture is prepared by mixing 10 μL 5× kinase buffer (250 mM HEPES, pH 7.5, 50 μM Na₃VO₄, 5 mM DTT, 10 mM MgCl₂, 50 mM MnCl₂, 0.05% BSA, 0.25% Tween-20 in water), 20 μL water, 5 μL of 4 μM biotinylated peptide substrate (Biot-Y397) in aqueous solution, 5 μL of test compound in DMSO and 5 μL of recombinant enzyme solution and incubated for 30 min. at room temperature. The enzyme reaction is started by addition of 5 μL of 5 μM ATP in water and the mixture is incubated for 3 hours at 37° C. The reaction is terminated by addition of 200 μL of detection mixture (1 nM Eu-PT66, 2.5 μg/mL SA-(SL)APC, 6.25 mM EDTA in dilution buffer), and the FRET signal from europium to allophycocyanin is measured by ARVOsx+L (Perkin Elmer) after 30 min. of incubation at room temperature. The ratio of fluorescence intensity of 665 nm to 615 nm is used as a FRET signal for data analysis in order to cancel the colour quenching effect by a test compound. The results are shown as percent inhibition of enzyme activity. DMSO and 0.5 M EDTA are used as a control of 0% and 100% inhibition, respectively. IC₅₀ values are determined by non-linear curve fit analysis using the OriginPro 6.1 program (OriginLab).

The Biot-Y397 peptide (Biotin-SETDDYAEIID ammonium salt) is designed to have the same amino acid sequence as the region from S392 to D402 of human (GenBank Accession Number L13616) and is prepared by standard methods.

Purified recombinant hexahistidine-tagged human FAK kinase domain is obtained in the following way: Full-length human FAK cDNA is isolated by PCR amplification from human placenta Marathon-Ready™ cDNA (Clontech, No. 7411-1) with the 5′ PCR primer (ATGGCAGCTGCTTACCTTGAC) and the 3′ PCR primer TCAGTGTGGTCTCGTCTGCCC) and subcloned into a pGEM-T vector (Promega, No. A3600). After digestion with AccIII, the purified DNA fragment is treated with Klenow fragment. The cDNA fragment is digested with BamHI and cloned into pFastBacHTb plasmid (Invitrogen Japan K.K., Tokyo) previously cut with BamHI and Stu I. The resultant plasmid, hFAK KD (M384-G706)/pFastBacHTb, is sequenced to confirm its structure. The resulting DNA encodes a 364 amino acid protein containing a hexahistidine tag, a spacer region and a rTEV protease cleavage site at the N-terminal and the kinase domain of FAK (Met384-Gly706) from position 29 to 351.

Donor plasmid is transposed into the baculovirus genome, using MaxEfficacy DH10Bac E. coli cells. Bacmid DNA is prepared by a simple alkaline lysis protocol described in the Bac-to-Bac® Baculovirus Expression system (Invitrogen). Sf9 insect cells are transfected based on the protocol provided by the vendor (CellFECTIN®, Invitrogen). The expression of FAK in each lysate is analysed by SDS-PAGE and Western blotting with anti-human FAK monoclonal antibody (clone #77 from Transduction Laboratories).

The virus clone that shows the highest expression is further amplified by infection to Sf9 cells. Expression in ExpresSF+® cells (Protein Sciences Corp., Meriden, Conn., USA) gives high level of protein with little degradation. Cell lysates are loaded onto a column of HiTrap™ Chelating Sepharose HP (Amersham Biosciences) charged with nickel sulfate and equilibrated with 50 mM HEPES pH 7.5, 0.5 M NaCl and 10 mM imidazole. Captured protein is eluted with increasing amounts of imidazole in HEPES buffer/NaCl, and further purified by dialysis in 50 mM HEPES pH 7.5, 10% glycerol and 1 mM DTT.
Suitable FAK inhibitors include compound of formula (XV), which are disclosed in WO 04/080980:

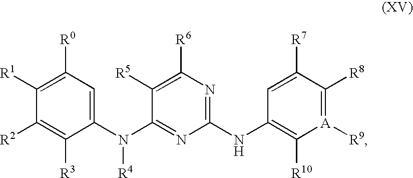

wherein
each of R⁰, R¹, R² and R³ independently is hydrogen, C₁-C₈-alkyl, C₂-C₈-alkenyl, C₂-C₈-alkinyl, C₃-C₈-cycloalkyl, C₃-C₈-cycloalkyl-C₁-C₈-alkyl, C₅-C₁₀-aryl-C₁-C₈-alkyl, hydroxy-C₁-C₈-alkyl, C₁-C₈-alkoxy-C₁-C₈-alkyl, amino-C₁-C₈-alkyl, halo-C₁-C₈-alkyl, unsubstituted or substituted C₅-C₁₀-aryl, unsubstituted or substituted 5- or 6-membered heterocyclyl comprising 1, 2 or 3 hetero atoms selected from N, O and S, hydroxy, C₁-C₈-alkoxy, hydroxy-C₁-C₈-alkoxy, C₁-C₈-alkoxy-C₁-C₈-alkoxy, halo-C₁-C₈-alkoxy, unsubstituted or substituted C₅-C₁₀-aryl-C₁-C₈-alkoxy, unsubstituted or substituted heterocyclyloxy, or unsubstituted or substituted heterocyclyl-C₁-C₈-alkoxy, unsubstituted or substituted amino, C₁-C₈-alkylthio, C₁-C₈-alkylsulfinyl, C₁-C₈-alkylsulfonyl, C₅-C₁₀-arylsulfonyl, halogen, carboxy, C₁-C₈-alkoxycarbonyl, unsubstituted or substituted carbamoyl, unsubstituted or substituted sulfamoyl, cyano or nitro, or
R⁰ and R¹, R¹ and R² and/or R² and R³ form, together with the carbon atoms to which they are attached, a 5- or 6-membered carbocyclic or heterocyclic ring comprising 0, 1, 2 or 3 heteroatoms selected from N, O and S;
R⁴ is hydrogen or C₁-C₈-alkyl;
each of R⁵ and R⁶ independently is hydrogen, C₁-C₈-alkyl, C₁-C₈-alkoxy-C₁-C₈-alkyl, halo-C₁-C₈-alkyl, C₁-C₈-alkoxy, halogen, carboxy, C₁-C₈-alkoxycarbonyl, unsubstituted or substituted carbamoyl, cyano or nitro; and
each of R⁷, R⁸, R⁹ and R¹⁰ independently is C₁-C₈-alkyl, C₂-C₈-alkenyl, C₂-C₈-alkinyl, C₃-C₈-cycloalkyl, C₃-C₈-cycloalkyl-C₁-C₈-alkyl, C₅-C₁₀-aryl-C₁-C₈-alkyl, hydroxy-C₁-C₈-alkyl, C₁-C₈-alkoxy-C₁-C₈-alkyl, amino-C₁-C₈-alkyl, halo-C₁-C₈-alkyl, unsubstituted or substituted C₅-C₁₀-aryl, unsubstituted or substituted 5- or 6-membered heterocyclyl comprising 1, 2 or 3 hetero atoms selected from N, O and S, hydroxy, C₁-C₈-alkoxy, hydroxy-C₁-C₈-alkoxy, C₁-C₈-alkoxy-C₁-C₈-alkoxy, halo-C₁-C₈-alkoxy, unsubstituted or substituted C₅-C₁₀-aryl-C₁-C₈-alkoxy, unsubstituted or substituted heterocyclyloxy, or unsubstituted or substituted heterocyclyl-C₁-C₈-alkoxy, unsubstituted or substituted amino, C₁-C₈-alkylthio, C₁-C₈-alkylsulfinyl, C₁-C₈-alkylsulfonyl, C₅-C₁₀-arylsulfonyl, halogen, carboxy, C₁-C₈-alkoxycarbonyl, unsubstituted or substituted carbamoyl, unsubstituted or substituted sulfamoyl, cyano or nitro, wherein R⁷, R⁸ and R⁹ independently of each other can also be hydrogen, or
R⁷ and R⁸, R⁸ and R⁹ and/or R⁹ and R¹⁰ form, together with the carbon atoms to which they are attached, a 5- or 6-membered carbocyclic or heterocyclic ring comprising 0, 1, 2 or 3 heteroatoms selected from N, O and S;
A is C or N;
   and salts thereof.

Specific examples of formula (XV) include
2-[5-bromo-2-(2-methoxy-5-morpholin-4-yl-phenylamino)-pyrimidin-4-ylamino]-N-methyl-benzenesulfonamide;
2-[5-chloro-2-(2-methoxy-4-morpholin-4-yl-phenylamino)-pyrimidin-4-ylamino]-N-methyl-benzamide;
N²-(4-[1,4′]bipiperidinyl-1′-yl-2-methoxy-phenyl)-5-chloro-N⁴-[2-(propane-1-sulfonyl)-phenyl]-pyrimidine-2,4-diamine; and
2-{5-chloro-2-[2-methoxy-4-(4-methyl-piperazin-1-yl)-phenylamino]-pyrimidin-4-ylamino}-N-isopropyl-benzenesulfonamide;
   or pharmaceutically acceptable salts thereof.

Additional FAK inhibitors are disclosed in WO 04/056786 to Pfizer; WO 03/024967 to Aventis; WO 01/064655 and WO 00/053595 to AstraZeneca; and WO 01/014402.

The Janus kinases, JAK1, JAK2, JAK3 and TYK2, are cytoplasmic protein tyrosine kinases which associate with multiple transmembrane receptors for chemokines (e.g., CCR2, CCR5, CCR7, CXCR4), interferons and cytokines (e.g., GM-CSF, erythropoietin, prolactin and interleukins (IL-2, IL-3, IL-4, IL-5, IL-6, IL-12 IL-13, etc.). Ligand binding to these receptors leads to activation of the associated JAK members, an essential event in the intracellular transmission of the receptor's signal. JAK activations results in phosphorylation of multiple downstream targets including the transcription factor family Signal Tranducer and Activator of Transcription (STAT). JAK activation regulates multiple processes, particularly within the haematopoietic compartment. Targeted disruption of JAK2 results in a embryonic lethal failure to produce mature erythrocytes, underlining the importance of JAK2 in mediating signaling from the erythropoietin growth factor receptor. Additional roles for JAK2 in prolactin signaling in the breast have also been delineated. JAK family members are also of importance in regulating inflammatory and immune responses, by controlling the development and homeostasis of lymphocytes and other immunomodulating cells. JAK3, an enzyme primarily expressed in T and B cells, plays a particularly critical role in the development of T cell and their ability to mount an immune response. Disruption of JAK3 signaling is associated with Severe Combined Immunodeficiency Syndromes (SCID) in both mice and humans.

JAK3 kinase inhibitors are, e.g., compounds having an IC₅₀ value <5 μM, preferably <1 μM, more preferably <0.1 μM in the following assays:

Interleukin-2 (IL-2) dependent proliferation assays with CTL/L and HT-2 cells: The IL-2 dependent mouse T cell lines CTL/L and HT-2 are cultured in RPMI 1640 (Gibco 52400-025) supplemented with 10% Fetal Clone I (HyClone), 50 μM 2-mercaptoethanol (31350-010), 50 μg/mL gentamycine (Gibco 15750-037), 1 mM sodium pyruvate (Gibco 11360-039), non-essential amino acids (Gibco 11140-035; 100×) and 250 U/mL mouse IL-2 (supernatant of X63-Ag8 transfected cells containing 50,000 U/mL mouse IL-2 according to Genzyme standard). Cultures are split twice a week 1:40.

Before use the cells are washed twice with culture medium without mouse IL-2. The proliferation assay is performed with 4000 CTL/L cells/well or 2500 HT-2 cells/well in flat-bottom 96-well tissue culture plates containing appropriate dilutions of test compounds in culture medium with 50 U/mL mouse IL-2. CTL/L cultures are incubated at 37° C. for 24 hours and HT-2 cultures are incubated for 48 hours. After addition of 1 μCi ³H-thymidine and a further overnight incubation cells are harvested onto fibre filters and radioactivity is counted.

Interleukin-2 dependent proliferation of human peripheral blood mononuclear cells: Human peripheral blood mononuclear cells are isolated on Ficoll from buffy coats with unknown HLA type (Blutspendezentrum, Kantonsspital, Basel, Switzerland). Cells are kept at 2×10⁷ cells/mL (90% FCS, 10% DMSO) in cryotubes (Nunc) in liquid nitrogen until use.

The cells are incubated for four days at 37° C. in a humidified CO₂ (7%) incubator in costar flasks at the concentration of 7×10⁵ cells/mL in culture medium containing RPMI 1640 (Gibco, Pacety, England) supplemented with Na-pyruvate (1 mM; Gibco), MEM non-essential amino acids and vitamins (Gibco), 2-mercaptoethanol (50 μM), L-glutamine (2 mM), gentamicin and penicillin/streptomycin (100 μg/mL; Gibco), bacto asparagine (20 μg/mL; Difco), human insulin (5 μg/mL; Sigma), human transferrin (40 μg/mL; Sigma), selected fetal calf serum (10%, Hyclone Laboratories, Logan, Utah) and 100 μg/mL phytohemagglutinine. Cells are washed twice in RPMI 1640 medium containing 10% FCS and incubated for 2 hours. After centrifugation, the cells are taken up in the culture medium mentioned above (without phytohemagglutinine) containing interleukin-2 (Chiron 200 U/mL), distributed in triplicates into flat-bottomed 96-well tissue culture plates (Costar #3596) at a concentration of 5×10⁴ cells/0.2 mL in the presence of appropriate concentrations of test compounds and incubated at 37° C. for 72 hours. 3H-thymidine (1 μCi/0.2 mL) was added for the last 16 hours of culture. Subsequently, cells are harvested and counted on a scintillation counter.

Suitable JAK kinase inhibitors include, e.g.,
Compounds as disclosed in U.S. Patent No. 2003/0073719A1, e.g., a compound of formula (XVI)

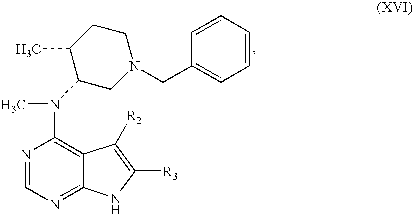

wherein
each of R₂ and R₃, independently, is selected from the group consisting of H, amino, halogen, OH, nitro, carboxy, C₂-C₆-alkenyl, C₂-C₆-alkynyl, CF₃, trifluoromethoxy, C₁-C₆-alkyl, C₁-C₆-alkoxy, C₃-C₆-cycloalkyl, wherein the alkyl, alkoxy or cycloalkyl groups are optionally substituted by one to three groups selected from halogen, OH, carboxy, amino, C₁-C₆-alkylthio, C₁-C₆-alkylamino, (C₁-C₆-alkyl)₂amino, C₅-C₉-heteroaryl, C₂-C₉-heterocycloalkyl, C₃-C₉-cycloalkyl or C₆-C₁₀-aryl, or
each of R₂ and R₃, independently, is C₃-C₁₀-cycloalkyl, C₃-C₁₀-cycloalkoxy, C₁-C₆-alkylamino, (C₁-C₆-alkyl)₂amino, C₆-C₁₀-arylamino, C₁-C₆-alkylthio, C₆-C₁₀-arylthio, C₁-C₆-alkylsulfinyl, C₆-C₁₀-arylsulfinyl, C₁-C₆-alkylsulfonyl, C₆-C₁₀-arylsulfonyl, C₁-C₆-acyl, C₁-C₆-alkoxy-CO—NH—, C₁-C₆-alkylamino-CO—, C₅-C₉-heteroaryl, C₂-C₉-heterocycloalkyl or C₆-C₁₀-aryl, wherein the heteroaryl, heterocycloalkyl and aryl groups are optionally substituted by one to three halogens, C₁-C₆-alkyl, C₁-C₆-alkyl-CO—NH—, C₁-C₆-alkoxy-CO—NH—, C₁-C₆-alkyl-CO—NH—C₁-C₆-alkyl, C₁-C₆-alkoxy-CO—NH—C₁-C₆-alkyl, C₁-C₆-alkoxy-CO—NH—C₁-C₆-alkoxy, carboxy, carboxy-C₁-C₆-alkyl, carboxy-C₁-C₆-alkoxy, benzyloxycarbonyl-C₁-C₆-alkoxy, C₁-C₆-alkoxycarbonyl-C₁-C₆-alkoxy, C₆-C₁₀-aryl, amino, amino-C₁-C₆-alkyl, C₂-C₇-alkoxycarbonylamino, C₆-C₁₀-aryl-C₂-C₇-alkoxycarbonylamino, C₁-C₆-alkylamino, (C₁-C₆-alkyl)₂amino, C₁-C₆-alkylamino-C₁-C₆-alkyl, (C₁-C₆-alkyl)₂amino-C₁-C₆-alkyl, hydroxy, C₁-C₆-alkoxy, carboxy, carboxy-C₁-C₆-alkyl, C₂-C₇-alkoxycarbonyl, C₂-C₇-alkoxycarbonyl-C₁-C₆-alkyl, C₁-C₆-alkoxy-CO—NH—, C₁-C₆-alkyl-CO—NH—, cyano, C₅-C₉-hetero-cycloalkyl, amino-CO—NH—, C₁-C₆-alkylamino-CO—NH—, (C₁-C₆-alkyl)₂amino-CO—NH—, C₆-C₁₀-arylamino-CO—NH—, C₅-C₉-heteroarylamino-CO—NH—, C₁-C₆-alkylamino-CO—NH—C₁-C₆-alkyl, (C₁-C₆-alkyl)₂amino-CO—NH—C₁-C₆-alkyl, C₆-C₁₀-arylamino-CO—NH—C₁-C₆-alkyl, C₅-C₉-heteroarylamino-CO—NH—C₁-C₆-alkyl, C₁-C₆-alkylsulfonyl, C₁-C₆-alkylsulfonylamino, C₁-C₆-alkylsulfonylamino-C₁-C₆-alkyl, C₆-C₁₀-arylsulfonyl, C₆-C₁₀-arylsulfonylamino, C₆-C₁₀-arylsulfonylamino-C₁-C₆-alkyl, C₁-C₆-alkylsulfonylamino, C₁-C₆-alkylsulfonylamino-C₁-C₆-alkyl, C₅-C₉-heteroaryl or C₂-C₉-heterocycloalkyl.

Examples of compound of (XVI) include, e.g.,
methyl-[(3R,4R)-4-methyl-1-(propane-1-sulfonyl)-piperidin-3-yl]-(7H-pyrrolo[2,3-d]pyrimidin-4-yl)-amine;
(3R,4R)-4-methyl-3-[methyl-(7H-pyrrolo[2-,3-d]pyrimidin-4-yl)-amino]-piperidine-1-carboxylic acid methyl ester;
3,3,3-trifluoro-1-{(3R,4R)-4-methyl-3-[methyl-(7H-pyrrolo[2,3-d]pyrimidin-4-yl)-amino]-piperidin-1-yl}-propan-1-one;
(3R,4R)-4-methyl-3-[methyl-(7H-pyrrolo[2,3-d]pyrimidin-4-yl)-amino]-piperidine-1-carboxylic acid dimethylamide;
{(3R,4R)-4-methyl-3-[methyl-(7H-pyrrolo[2,3-d]pyrimidin-4-yl)-amino]-piperidine-1-carbonyl}-amino)-acetic acid ethyl ester;
3-{(3R,4R)-4-methyl-3-[methyl-(7H-pyrrolo[2,3-d]pyrimidin-4-yl)-amino]-piperidin-1-yl}-3-oxo-propionitrile;
3,3,3-trifluoro-1-{(3R,4R)-4-methyl-3-[methyl-(5-methyl-7H-pyrrolo[2,3-d]pyrimidin-4-yl)-amino]-piperidin-1-yl}-propan-1-one;
1-{(3R,4R)-4-methyl-3-[methyl-(7H-pyrrolo[2,3-d]pyrimidin-4-yl)-amino]piperidin-1-yl}-but-3-yn-1-one;
1-{(3R,4R)-3-[(5-chloro-7H-pyrrolo[2,3-d]pyrimidin-4-yl)-methyl-amino]-4-methyl-piperidin-1-yl}-propan-1-one;
1-{(3R,4R)-3-[(5-fluoro-7H-pyrrolo[2,3-d]pyrimidin-4-yl)-methyl-amino]-4-methyl-piperidin-1-yl}-propan-1-one;
(3R,4R)—N-cyano-4-methyl-3-[methyl-(7H-pyrrolo[2,3-d]pyrimidin-4-yl)-amino]-N′-propyl-piperidine-1-carboxamidine; or
(3R,4R)—N-cyano-4,N′,N′-trimethyl-3-[methyl-(7H-pyrrolo[2,3-d]pyrimidin-4-yl)-amino]-piperidine-1-carboxamidine.
   Additional JAK inhibitors include compounds as disclosed in WO 02/092571, e.g., a compound of formula (XVII)

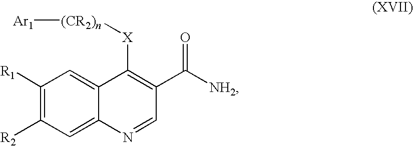

wherein
X is NR₃ or O;
n is 0 or 1;
Ar₁ is selected from phenyl, tetrahydronaphthenyl, indolyl, pyrazolyl, dihydroindenyl, 1-oxo-2,3-dihydroindenyl or indazolyl, each of which can be optionally substituted by one or more groups selected from halogen, hydroxy, cyano, C₁-C₈-alkoxy, CO₂R⁸, CONR⁹R¹⁰, C₁-C₈-alkyl-O—C₁-C₈-alkyl, C₁-C₈-alkyl-NR⁸—C₁-C₈-alkyl, C₁-C₈-alkyl-CONR⁸—C₁-C₈-alkyl, C₁-C₈-alkyl-CONR⁹R¹⁰, NR₈CO—C₁-C₈-alkyl, C₁-C₈-thioalkyl, C₁-C₈-alkyl (itself optionally substituted by one or more OH or cyano or fluorine) or C₁-C₈-alkoxy;
R groups are independently is hydrogen or C₁-C₈-alkyl;
R₁ and R₂, independently, is selected from H, halogen, nitro, cyano, C₁-C₈-alkyl, C₁-C₈-alkoxy, OH, aryl, Y(CR¹¹₂)ₚNR₄R₅, Y(CR¹¹₂)ₚCONR₄R₅, Y(CR¹¹₂)ₚCO₂R₆, Y(CR¹¹₂)ₚOR₆ or Y(CR¹¹₂)ₚR₆, or
R₁ and R₂ are linked together as —OCHO— or —OCH₂CH₂O—;
   each R¹¹, independently, is H, C₁-C₈-alkyl, hydroxy or halogen;
   p is 0, 1, 2, 3, 4 or 5;
   R₃ is H or C₁-C₈-alkyl;
   Y is oxygen, CH₂ or NR₇R₃ is hydrogen or C₁-C₈-alkyl;
   each of R₄ and R₅, independently, is H, C₁-C₈-alkyl, or
   R₄ and R₅, together with the nitrogen atom to which they are attached, form a 4- to 7-membered saturated or aromatic heterocyclic ring system optionally containing a further O, S or NR₆, or
   one of R₄ and R₅ is H or C₁-C₈-alkyl and the other is a 5- or 6-membered heterocyclic ring system optionally containing a further O, S or N atom;
   R₆ is H, C₁-C₈-alkyl, phenyl or benzyl;
   R₇ is H or C₁-C₈-alkyl;
   R₈ is H or C₁-C₈-alkyl;
   each of R₉ and R₁₀, independently, is hydrogen or C₁-C₈-alkyl;
      and pharmaceutically acceptable salts thereof.
Examples of additional JAK inhibitors include compounds as disclosed in U.S. Patent No. 2002/0055514 A1, e.g., a compound of formula (XVIII)

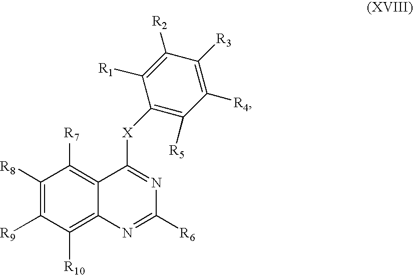

wherein
X is NH, NR₁₁, S, O CH₂ or R₁₁CH, wherein R₁₁ is H, C₁-C₄-alkyl or C₁-C₄-alkanoyl;
each of R₁-R₈, independently, is H, halogen, OH, mercapto, amino, nitro, C₁-C₄-alkyl, C₁-C₄-alkoxy or C₁-C₄alkylthio, wherein 2 of R₁-R₅, together with the phenyl ring to which they are attached, may optionally form a fused ring, e.g., forming a naphthyl or a tetrahydronaphthyl ring; and further wherein the ring formed by the two adjacent groups of R₁-R₅ may optionally be substituted by 1, 2, 3 or 4 halogen, hydroxy, mercapto, amino, nitro, C₁-C₄-alkyl, C₁-C₄alkoxy or C₁-C₄-alkylthio, and provided that at least one of R₂-R₅ is OH; and
each of R₉ and R₁₀, independently, is H, halogen, C₁-C₄-alkyl, C₁-C₄-alkoxy or C₁-C₄-alkanoyl, or
R₉ and R₁₀, together, are methylenedioxy;
   or a pharmaceutically acceptable salt thereof, and provided that at least one of R₂-R₅ is OH.
Additional JAK inhibitors include compounds as disclosed in WO 04/052359, e.g., a compound of formula (XIX)

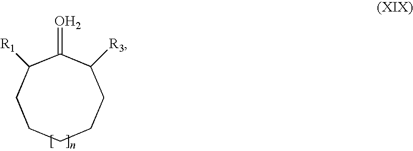

wherein
n is 1, 2, 3, 4 or 5;
R₁ is H, CH₃ or CH₂N(CH₃)₂; and
R₃ is CH₂N(CH₃)₂.

The compounds of formulae (XVI)-(XIX) may exist in free or salt form. Examples of pharmaceutically acceptable salts of the compounds of the formulae (XVI)-(XIX) include salts with inorganic acids, such as hydrochloride; salts with organic acids, such as acetate or citric acid, or, when appropriate, salts with metals, such as sodium or potassium; salts with amines, such as triethylamine; and salts with dibasic amino acids, such as lysine.
Additional JAK inhibitors include compounds as disclosed in WO 03/048162, e.g., amorphous and crystalline forms of 3-{(3R,4R)-4-methyl-3-[methyl-(7H-pyrrolo[2,3-d]pyrimidin-4-yl)-amino]-piperidin-1-yl}-3-oxo-propionitrile mono citrate salt.
Additional JAK inhibitors include compounds as disclosed in WO 01/42246 and WO 02/096909, e.g., a compound of formula (XX)

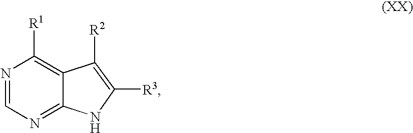

or the pharmaceutically acceptable salt thereof,
wherein
R¹ is a group of the formula (XXI)

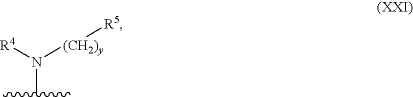

wherein
   y is 0, 1 or 2;
   R⁴ is selected from the group consisting of hydrogen, C₁-C₆-alkyl, C₁-C₆-alkylsulfonyl, C₂-C₆-alkenyl, C₂-C₆-alkynyl, wherein the alkyl, alkenyl and alkynyl groups are optionally substituted by deuterium, hydroxy, amino, trifluoromethyl, C₁-C₄-alkoxy, C₁-C₆-acyloxy, C₁-C₆-alkylamino, (C₁-C₆-alkyl)₂amino, cyano, nitro, C₂-C₆-alkenyl, C₂-C₆-alkynyl or C₁-C₆-acylamino, or
   R⁴ is C₃-C₁₀-cycloalkyl, wherein the cycloalkyl group is optionally substituted by deuterium, hydroxy, amino, trifluoromethyl, C₁-C₆-acyloxy, C₁-C₆-acylamino, C₁-C₆-alkylamino, (C₁-C₆-alkyl)₂amino, cyano, cyano-C₁-C₆-alkyl, trifluoromethyl-C₁-C₆-alkyl, nitro, nitro-C₁-C₆-alkyl or C₁-C₆-acylamino;
   R⁵ is C₂-C₉-heterocycloalkyl, wherein the heterocycloalkyl groups must be substituted by one to five carboxy, cyano, amino, deuterium, hydroxy, C₁-C₆-alkyl, C₁-C₆-alkoxy, halo, C₁-C₆-acyl, C₁-C₆-alkylamino, amino-C₁-C₆-alkyl, C₁-C₆-alkoxy-CO—NH, C₁-C₆-alkylamino-CO—, C₂-C₆-alkenyl, C₂-C₆-alkynyl, C₁-C₆-alkylamino, amino-C₁-C₆-alkyl, hydroxy-C₁-C₆-alkyl, C₁-C₆-alkoxy-C₁-C₆-alkyl, C₁-C₆-acyloxy-C₁-C₆-alkyl, nitro, cyano-C₁-C₆-alkyl, halo-C₁-C₆-alkyl, nitro-C₁-C₆-alkyl, trifluoromethyl, trifluoromethyl-C₁-C₆-alkyl, C₁-C₆-acylamino, C₁-C₆-acylamino-C₁-C₆-alkyl, C₁-C₆-alkoxy-C₁-C₆-acylamino, amino-C₁-C₆-acyl, amino-C₁-C₆-acyl-C₁-C₆-alkyl, C₁-C₆-alkylamino-C₁-C₆-acyl, (C₁-C₆-alkyl)₂amino-C₁-C₆-acyl, R¹⁵R¹⁶N—CO—O—, R¹⁵R¹⁶N—CO—C₁-C₆-alkyl-, C₁-C₆-alkyl-S(O)ₘ, R¹⁵R¹⁶NS(O)ₘ, R¹⁵R¹⁶NS(O)ₘ—C₁-C₆-alkyl, R¹⁵S(O)ₘR¹⁶N, R¹⁵S(O)ₘR¹⁶N—C₁-C₆-alkyl,
   wherein
      m is 0, 1 or 2; and
      R¹⁵ and R¹⁶ are each independently selected from hydrogen or C₁-C₆-alkyl,
   or a group of the formula (XXII)

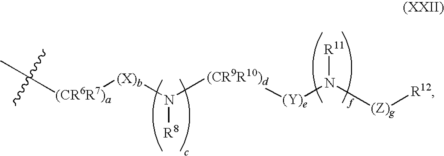

wherein
   a is 0, 1, 2, 3 or 4;
   b, c, e, f and g are each independently 0 or 1;
   d is 0, 1, 2 or 3;
   X is S(O)ₙ, wherein n is 0, 1 or 2, oxygen, carbonyl or —C(═N-cyano)-;
   Y is S(O)ₙ, wherein n is 0, 1 or 2 or carbonyl;
   Z is carbonyl, C(O)O—, C(O)NR— or S(O)ₙ, wherein n is 0, 1 or 2;
   R⁶, R⁷, R⁸, R⁹, R¹⁰ and R¹¹ are each independently selected from the group consisting of hydrogen or C₁-C₆-alkyl optionally substituted by deuterium, hydroxy, amino, trifluoromethyl, C₁-C₆-acyloxy, C₁-C₆-acylamino, C₁-C₆-alkylamino, (C₁-C₆-alkyl)₂amino, cyano, cyano-C₁-C₆-alkyl, trifluoromethyl-C₁-C₆-alkyl-, nitro, nitro-C₁-C₆-alkyl or C₁-C₆-acylamino;
   R¹² is carboxy, cyano, amino, oxo, deuterium, hydroxy, trifluoromethyl, C₁-C₆-alkyl, trifluoromethyl-C₁-C₆-alkyl, C₁-C₆-alkoxy, halo, C₁-C₆-acyl, C₁-C₆-alkylamino, (C₁-C₆-alkyl)₂amino, amino-C₁-C₆-alkyl, C₁-C₆-alkoxy-CO—NH, C₁-C₆-alkylamino-CO—, C₂-C₆-alkenyl, C₂-C₆-alkynyl, C₁-C₆-alkylamino, hydroxy-C₁-C₆-alkyl, C₁-C₆-alkoxy-C₁-C₆-alkyl, C₁-C₆-acyloxy-C₁-C₆-alkyl, nitro, cyano-C₁-C₆-alkyl, halo-C₁-C₆-alkyl, nitro-C₁-C₆-alkyl, trifluoromethyl, trifluoromethyl-C₁-C₆-alkyl, C₁-C₆-acylamino, C₁-C₆-acylamino-C₁-C₆-alkyl, C₁-C₆-alkoxy-C₁-C₆-acylamino, amino-C₁-C₆-acyl, amino-C₁-C₆-acyl-C₁-C₆-alkyl, C₁-C₆-alkylamino-C₁-C₆-acyl, (C₁-C₆-alkyl)₂amino-C₁-C₆-acyl, R¹⁵R¹⁶N—CO—O—, R¹⁵R¹⁶N—CO—C₁-C₆-alkyl-, R¹⁵C(O)NH, R¹⁵OC(O)NH, R¹⁵NHC(O)NH, C₁-C₆-alkyl-S(O)ₘ, C₁-C₆-alkyl-S(O)ₘ—C₁-C₆-alkyl, R¹⁵R¹⁶NS(O)ₘ, R¹⁵R¹⁶NS(O)ₘ—C₁-C₆-alkyl, R¹⁵S(O)ₘR¹⁶N, R¹⁵S(O)ₘR¹⁶N—C₁-C₆-alkyl,
   wherein
      m is 0, 1 or 2; and
      R¹⁵ and R¹⁶ are each independently selected from hydrogen or C₁-C₆-alkyl;
R² and R³ are each independently selected from the group consisting of hydrogen, deuterium, amino, halo, hydroxy, nitro, carboxy, C₂-C₆-alkenyl, C₂-C₆-alkynyl, trifluoromethyl, trifluoromethoxy, C₁-C₆-alkyl, C₁-C₆-alkoxy, C₃-C₁₀-cycloalkyl, wherein the alkyl, alkoxy or cycloalkyl groups are optionally substituted by one to three groups selected from halo, hydroxy, carboxy, amino-C₁-C₆-alkylthio, C₁-C₆-alkylamino, (C₁-C₆-alkyl)₂amino, C₅-C₉-heteroaryl, C₂-C₉-heterocycloalkyl, C₃-C₉-cycloalkyl or C₆-C₁₀-aryl, or
R² and R³ are each independently C₃-C₁₀-cycloalkyl, C₃-C₁₀-cycloalkoxy, C₁-C₆-alkylamino, (C₁-C₆-alkyl)₂amino, C₆-C₁₀-arylamino, C₁-C₆-alkylthio, C₆-C₁₀-arylthio, C₁-C₆-alkylsulfinyl, C₆-C₁₀-arylsulfinyl, C₁-C₆-alkylsulfonyl, C₆-C₁₀-arylsulfonyl, C₁-C₆-acyl, C₁-C₆-alkoxy-CO—NH—, C₁-C₆-alkyamino-CO—, C₅-C₉-heteroaryl, C₂-C₉-heterocycloalkyl or C₆-C₁₀-aryl, wherein the heteroaryl, heterocycloalkyl and aryl groups are optionally substituted by one to three halo, C₁-C₆-alkyl, C₁-C₆-alkyl-CO—NH—, C₁-C₆-alkoxy-CO—NH—, C₁-C₆-alkyl-CO—NH—C₁-C₆-alkyl, C₁-C₆-alkoxy-CO—NH—C₁-C₆-alkyl, C₁-C₆-alkoxy-CO—NH—C₁-C₆-alkoxy, carboxy, carboxy-C₁-C₆-alkyl, carboxy-C₁-C₆-alkoxy, benzyloxycarbonyl-C₁-C₆-alkoxy, C₁-C₆-alkoxycarbonyl-C₁-C₆-alkoxy, C₆-C₁₀-aryl, amino, amino-C₁-C₆-alkyl, C₁-C₆-alkoxycarbonylamino, C₆-C₁₀-aryl-C₁-C₆-alkoxycarbonylamino, C₁-C₆-alkylamino, (C₁-C₆-alkyl)₂amino, C₁-C₆-alkylamino-C₁-C₆-alkyl, (C₁-C₆-alkyl)₂amino-C₁-C₆-alkyl, hydroxy, C₁-C₆-alkoxy, carboxy, carboxy-C₁-C₆-alkyl, C₁-C₆-alkoxycarbonyl, C₁-C₆-alkoxycarbonyl-C₁-C₆-alkyl, C₁-C₆-alkoxy-CO—NH—, C₁-C₆-alkyl-CO—NH—, cyano, C₅-C₉-heterocycloalkyl, amino-CO—NH—, C₁-C₆-alkylamino-CO—NH—, (C₁-C₆-alkyl)₂amino-CO—NH—, C₆-C₁₀-arylamino-CO—NH—, C₅-C₉-heteroarylamino-CO—NH—, C₁-C₆-alkylamino-CO—NH—C₁-C₆-alkyl, (C₁-C₆)alkyl)₂amino-CO—NH—C₁-C₆-alkyl, C₆-C₁₀-arylamino-CO—NH—C₁-C₆-alkyl, C₅-C₉-heteroarylamino-CO—NH—C₁-C₆-alkyl, C₁-C₆-alkylsulfonyl, C₁-C₆-alkylsulfonylamino, C₁-C₆-alkylsulfonylamino-C₁-C₆-alkyl, C₆-C₁₀-arylsulfonyl, C₆-C₁₀-arylsulfonylamino, C₆-C₁₀-arylsulfonylamino-C₁-C₆-alkyl, C₁-C₆-alkylsulfonylamino, C₁-C₆-alkylsulfonylamino-C₁-C₆-alkyl, C₅-C₉-heteroaryl or C₂-C₉-heterocycloalkyl.
   Additional JAK inhibitors include compounds as disclosed in WO 02/060492, to Cytopic, e.g., a compound of formula (XXIII)

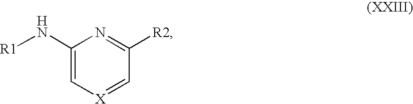

or pharmaceutically acceptable salts, hydrates, solvates, crystal forms or diastereomers thereof, wherein
X is either carbon or nitrogen;
R1 is C₁-C₁₀-allyl, C₂-C₁₀-alkenyl, C₂-C₁₀-alkynyl, C₂-C₁₀-allylaryl, aryl or heterocyclyl, or
R1 with N may form a substituted or unsubstituted heterocyclyl, wherein the allyl, alkenyl, alkynyl, allylaryl, aryl, and heterocyclyl, is optionally substituted with one to three members selected from the group consisting of halo, amino, hydroxy, hydroxyalkyl, alkylamide, arylamide, hydroxyallylamide, nitrilo, aminoalkylamide, nitriloaryl, alkoxy (in particular, methoxy), heterocyclic alkyl in which heterocycle is a 5- to 7-membered ring and in which the hetero atom is O, N or S;
R2 is selected from C₁-C₁₀-allyl, C₂-C₁₀-alkenyl, C₂-C₁₀-alkynyl, C₂-C₁₀-allylaryl, aryl, halo, OH, or 6- to 7-membered heterocyclyl, wherein the alkyl, alkenyl, alkynyl, allylaryl, aryl and heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular, methoxy), heterocyclic alkyl, in which heterocycle is a 5- to 7-membered ring and in which the hetero atom is O, N or S.
   Additional JAK inhibitors include compounds also disclosed in WO 02/060492, to cytopic, e.g., a compound of formula (XXIV)

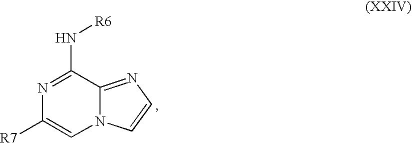

or pharmaceutically acceptable salts, hydrates, solvates, crystal forms or diastereomers thereof, wherein
R6 is C₁-C₁₀-allyl, C₂-C₁₀-alkenyl, C₂-C₁₀-alkynyl, C₂-C₁₀-allylaryl, aryl or heterocyclyl; and
R7 is C₁-C₁₀-allyl, C₂-C₁₀-alkenyl, C₂-C₁₀-alkynyl, C₂-C₁₀-allylaryl, aryl, halo, OH or heterocyclyl, wherein the alkyl, alkenyl, alkynyl, alkylaryl, aryl and heterocyclyl, is optionally substituted with one to three members selected from the group consisting of halo, amino, hydroxy, hydroxyalkyl, alkylamide, arylamide, hydroxyalkylamide, nitrilo, aminoalkylamide, nitriloaryl, alkoxy (in particular, methoxy), heterocyclic alkyl in which heterocycle is a 5- to 7-membered ring and in which the hetero atom is O, N or S.

Preferred JAK kinase inhibitors include, e.g.,
N-benzyl-3,4-dihydroxy-benzylidene-cyanoacetamide α-cyano-(3,4-dihydroxy)-N′-benzylcinnamamide (Tyrphostin AG 490); prodigiosin 25-C (PNU156804);
[4-(4′-hydroxyphenyl)-amino-6,7-dimethoxyquinazoline] (WHI-P131);
[4-(3′-bromo-4′-hydroxylphenyl)-amino-6,7-dimethoxyquinazoline] (WHI-P154);
[4-(3′,5′-dibromo-4′-hydroxylphenyl)-amino-6,7-dimethoxyquinazoline] WHI-P97; and
3-{(3R,4R)-4-methyl-3-[methyl-(7H-pyrrolo[2,3-d]pyrimidin-4-yl)-amino]-piperidin-1-yl}-3-oxo-propionitrile,
   in free form or in salt form, e.g., mono-citrate (also called CP-690,550).

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g., solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In accordance with the particular findings of the present invention, there is provided:
1. A pharmaceutical combination comprising:
   a) at least one agent selected from Bcr-Abl, Flt-3, RAF and FAK kinase inhibitors; and
   b) at least one JAK kinase inhibitor.
2. A method for treating or preventing proliferative disease in a subject in need thereof, comprising co-administration to said subject, e.g., concomitantly or in sequence, of a therapeutically effective amount of at least one agent selected from Bcr-Abl, Flt-3, RAF and FAK kinase inhibitors and at least one JAK3 kinase inhibitor, e.g., as disclosed above.
Examples of proliferative diseases include e.g. tumors, psoriasis, restenosis, sclerodermitis and fibrosis.
3. A pharmaceutical combination as defined under 1) above, e.g., for use in a method as defined under 2) above.
4. A pharmaceutical combination as defined under 1) above for use in the preparation of a medicament for use in a method as defined under 2) above.

Utility of the combination of the invention in a method as hereinabove specified, may be demonstrated in animal test methods as well as in clinic, for example in accordance with the methods hereinafter described.

### A. Combined Treatment

Suitable clinical studies are, e.g., open label, dose escalation studies in patients with proliferative diseases. Such studies prove in particular the synergism of the active ingredients of the combination of the invention. The beneficial effects on psoriasis or multiple sclerosis can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side-effects, an improved quality of life or a decreased morbidity, compared with a monotherapy applying only one of the pharmaceutically active ingredients used in the combination of the invention.

A further benefit is that lower doses of the active ingredients of the combination of the invention can be used, e.g., that the dosages need not only often be smaller but are also applied less frequently, which may diminish the incidence or severity of side effects. This is in accordance with the desires and requirements of the patients to be treated.

The terms “co-administration” or “combined administration” or the like as utilized, herein, are meant to encompass administration of the selected therapeutic agents to a single patient, and are intended to include treatment regimens in which the agents are not necessarily administered by the same route of administration or at the same time.

It is one objective of this invention to provide a pharmaceutical composition comprising a quantity, which is jointly therapeutically effective at targeting or preventing proliferative diseases a combination of the invention. In this composition, agent (a) and agent (b) may be administered together, one after the other or separately in one combined unit dosage form or in two separate unit dosage forms. The unit dosage form may also be a fixed combination.

The pharmaceutical compositions for separate administration of agent (a) and agent (b) or for the administration in a fixed combination, i.e., a single galenical composition comprising at least two combination partners (a) and (b), according to the invention may be prepared in a manner known per se and are those suitable for enteral, such as oral or rectal; and parenteral administration to mammals (warm-blooded animals) including humans, comprising a therapeutically effective amount of at least one pharmacologically active combination partner alone, e.g., as indicated above, or in combination with one or more pharmaceutically acceptable carriers or diluents, especially suitable for enteral or parenteral application.

Suitable pharmaceutical compositions contain, e.g., from about 0.1% to about 99.9%, preferably from about 1% to about 60%, of the active ingredient(s). Pharmaceutical preparations for the combination therapy for enteral or parenteral administration are, e.g., those in unit dosage forms, such as sugar-coated tablets, tablets, capsules or suppositories, or ampoules. If not indicated otherwise, these are prepared in a manner known per se, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Daily dosages for agent (a) or (b) or will, of course, vary depending on a variety of factors, for example the compound chosen, the particular condition to be treated and the desired effect. In general, however, satisfactory results are achieved on administration of agent (a) at daily dosage rates of the order of ca. 0.03-5 mg/kg per day, particularly 0.1-5 mg/kg per day, e.g., 0.1-2.5 mg/kg per day, as a single dose or in divided doses. Agents (a) and (b) may be administered by any conventional route, in particular enterally, e.g., orally, e.g., in the form of tablets, capsules, drink solutions or parenterally, e.g., in the form of injectable solutions or suspensions. Suitable unit dosage forms for oral administration comprise from ca. 0.02-50 mg active ingredient, usually 0.1-30 mg, e.g., agent (a) or (b), together with one or more pharmaceutically acceptable diluents or carriers therefore.

Agent (b) may be administered to a human in a daily dosage range of 0.5-1000 mg. Suitable unit dosage forms for oral administration comprise from ca. 0.1-500 mg active ingredient, together with one or more pharmaceutically acceptable diluents or carriers therefore.

The administration of a pharmaceutical combination of the invention results not only in a beneficial effect, e.g., a synergistic therapeutic effect, e.g., with regard to inhibiting the unregulated proliferation of haematological stem cells or slowing down the progression of leukemias, such as CML or AML, or the growth of tumors, but also in further surprising beneficial effects, e.g., less side effects, an improved quality of life or a decreased morbidity, compared to a monotherapy applying only one of the pharmaceutically active ingredients used in the combination of the invention.

A further benefit is that lower doses of the active ingredients of the combination of the invention can be used, e.g., that the dosages need not only often be smaller but are also applied less frequently, or can be used in order to diminish the incidence of side effects. This is in accordance with the desires and requirements of the patients to be treated.

The utility of the combinations of the present invention inhibiting the proliferation of leukemia cells for the treatment of leukemia can be demonstrated, e.g., in the proliferation test using Bcr-Abl transfected 32D cells as follows:

Bcr-Abl-transfected 32D cells (32D pGD p210 Bcr-Abl; Bazzoni et al., J Clin Invest, Vol. 98, No. 2, pp. 521-528 (1996)) are cultured in RPMI 1640 (BioConcept, Allschwil, Switzerland; Cat. No. 1-41F01), 10% fetal calf serum, 2 mM glutamine. 10000 cells in 50 μL per well are seeded into flat bottom 96-well tissue culture plates. Complete medium alone (for controls) or serial three-fold dilutions of compounds are added in triplicates to a final volume of 100 μL and the cells are incubated at 37° C., 5% CO₂ for 65-72 hours. The cell proliferation reagent WST-1 (Roche Diagnostics GmbH; Cat. No. 1 664 807) is added at 10 μL per well followed by 2 hours incubation at 37° C. Color development, depending on the amount of living cells, is measured at 440 nm. The effect for each compound is calculated as percent inhibition of the value (OD₄₄₀) obtained for the control cells (100%) and plotted against the compound concentrations. The IC₅₀s are calculated from the dose response curves by graphic extrapolation.

Compounds inhibiting the growth of 32D-Bcr-Abl cells can be further tested on IL-3 dependent 32D wt cells to prove the specificity of the compounds for the Bcr-Abl kinase and to exclude compound toxicity.

The proliferation test using Bcr-Abl transfected 32D cells with a COMBINATION OF THE INVENTION is carried out as described above with the following changes. Two combination partners are mixed in fixed ratios. Three-fold serial dilutions of this mixture or the combination partners alone are added to the cells seeded in 96-well tissue culture plates as described above. The effects on 32D-Bcr-Abl cell proliferation of a COMBINATION OF THE INVENTION is evaluated and compared with the effects of the single combination partners using CalcuSyn, a dose-effect analyzer software for single and multiple drugs (distributed by Biosoft, Cambridge).

Preferred are combinations wherein the JAK inhibitor is selected from the group consisting of:
N-benzyl-3,4-dihydroxy-benzylidene-cyanoacetamide α-cyano-(3,4-dihydroxy)-]N-benzylcinnamamide (Tyrphostin AG 490); prodigiosin 25-C (PNU156804);
[4-(4′-hydroxyphenyl)-amino-6,7-dimethoxyquinazoline] (WHI-P131);
[4-(3′-bromo-4′-hydroxylphenyl)-amino-6,7-dimethoxyquinazoline] (WHI-P154);
[4-(3′,5′-dibromo-4′-hydroxylphenyl)-amino-6,7-dimethoxyquinazoline] WHI-P97; and
3-{(3R,4R)-4-methyl-3-[methyl-(7H-pyrrolo[2,3-d]pyrimidin-4-yl)-amino]-piperidin-1-yl}-3-oxo-propionitrile,
   in free form or in salt form, e.g., mono-citrate (also called CP-690,550), and combinations thereof.

In another preferred embodiment, the Bcr-Abl, Flt-3 and RAF kinase inhibitor is selected from:
N-{5-[4-(4-methyl-piperazino-methyl)-benzoylamido]-2-methyl-phenyl}-4-(3-pyridyl)-2-pyrimidine-amine;
4-methyl-3-[[4-(3-pyridinyl)-2-pyrimidinyl]amino]-N-[5-(4-methyl-1H-imidazol-1-yl)-3-(trifluoromethyl)phenyl]benzamide;
N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide;
1-[4-(4-ethyl-piperazinyl-1-ylmethyl)-3-trifluoromethyl-phenyl]-3-[4-(6-methylamino-pyrimidin-4-yloxy-phenyl]-urea;
1-[4-(2-amino-pyrimidin-4-yloxy)-phenyl]-3-[4-(4-ethyl-piperazin-1-ylmethyl)-3-trifluoromethyl-phenyl]-urea;
1-[4-(2-amino-pyrimidin-4-yloxy)-phenyl]-3-[4-(4-methyl-piperazin-1-ylmethyl)-3-trifluoromethyl-phenyl]-urea;
(4-tert-butyl-phenyl)-(4-pyridin-4-ylmethyl-isoquinolin-1-yl)-amine;
[4,7′]biisoquinolinyl-1-yl-4-(tert-butyl-phenyl)-amine;
(4-tert-butyl-phenyl)-(4-quinazolin-6-yl-isoquinolin-1-yl)-amine;
[4,7′]biisoquinolinyl-1-yl-(2-tert-butyl-pyrimidin-5-yl)-amine;
2-[5-chloro-2-(2-methoxy-4-morpholin-4-yl-phenylamino)-pyrimidin-4-ylamino]-N-methyl-benzamide;
N3-[4-(4-morpholin-4-yl-cyclohexyl)-phenyl]-1-pyridin-2-yl-1H-[1,2,4]triazole-3,5-diamine;
   and combinations thereof.

### B. Diseases to be Treated

The term “proliferative disease” includes but is not restricted to tumors, psoriasis, restenosis, sclerodermitis and fibrosis.

The term haematological malignancy, refers in particular to leukemias, especially those expressing Bcr-Abl, c-Kit or Flt-3, and includes, but is not limited to, chronic myelogenous leukemia and acute lymphocyte leukemia (ALL), especially the Philadelphia chromosome positive acute lymphocyte leukemia (Ph+ALL), as well as STI571-resistant leukemia.

The term “a solid tumor disease” especially means ovarian cancer, breast cancer, cancer of the colon and generally the gastrointestinal tract, cervix cancer, lung cancer, e.g., small-cell lung cancer and non-small-cell lung cancer, head and neck cancer, bladder cancer, cancer of the prostate or Kaposi's sarcoma.

The combinations according to the invention, that inhibit the protein kinase activities mentioned, especially tyrosine protein kinases mentioned above and below, can therefore be used in the treatment of protein kinase dependent diseases. Protein kinase dependent diseases are especially proliferative diseases, preferably benign or especially malignant tumours (e.g., carcinoma of the kidneys, liver, adrenal glands, bladder, breast, stomach, ovaries, colon, rectum, prostate, pancreas, lungs, vagina or thyroid, sarcoma, glioblastomas and numerous tumours of the neck and head, as well as leukemias). They are able to bring about the regression of tumours and to prevent the formation of tumor metastases and the growth of (also micro)metastases. In addition, they can be used in epidermal hyperproliferation (e.g., psoriasis), in prostate hyperplasia, and in the treatment of neoplasias, especially of epithelial character, for example mammary carcinoma. It is also possible to use the combinations of the present invention in the treatment of diseases of the immune system insofar as several or, especially, individual tyrosine protein kinases are involved; furthermore, the combinations of the present invention can be used also in the treatment of diseases of the central or peripheral nervous system where signal transmission by at least one tyrosine protein kinase, especially selected from those mentioned specifically, is involved.

Flt-3 (FMD-like tyrosine kinase) is especially expressed in hematopoietic progenitor cells and in progenitors of the lymphoid and myeloid series. Aberrant expression of the Flt-3 gene has been documented in both adult and childhood leukemias including AML (acute myelogenous leukemia), AML with trilineage myelodysplasia (AML/TMDS), ALL, CML (chronic myelogenous leukemia) and myelodysplastic syndrome (MDS), which are therefore the preferred diseases to be treated with compounds of the formula (I). Activating mutations in Flt-3 have been found in approximately 25-30% of patients with AML. Thus, there is accumulating evidence for the role of Flt-3 in human leukemias, and the combinations of the present invention, as Flt-3 inhibitors are especially of use in the therapy of this type of diseases (see Tse et al., Leukemia, Vol. 15, No. 7, pp. 1001-1010 (2001); Tomoki et al., Cancer Chemother Pharmacol, Vol. 48, Suppl. 1, pp. S27-S30 (2001); Birkenkamp et al., Leukemia, Vol. 15, No. 12, pp. 1923-1921 (2001); Kelly et al., Neoplasia, Vol. 99, No. 1, pp. 310-318 (2002)).

In CML, a reciprocally balanced chromosomal translocation in hematopoietic stem cells (HSCs) produces the Bcr-Abl hybrid gene. The latter encodes the oncogenic Bcr-Abl fusion protein. Whereas ABL encodes a tightly regulated protein tyrosine kinase, which plays a fundamental role in regulating cell proliferation, adherence and apoptosis, the Bcr-Abl fusion gene encodes as constitutively activated kinase, which transforms HSCs to produce a phenotype exhibiting deregulated clonal proliferation, reduced capacity to adhere to the bone marrow stroma and a reduces apoptotic response to mutagenic stimuli, which enable it to accumulate progressively more malignant transformations. The resulting granulocytes fail to develop into mature lymphocytes and are released into the circulation, leading to a deficiency in the mature cells and increased susceptibility to infection. ATP-competitive inhibitors of Bcr-Abl have been described which prevent the kinase from activating mitogenic and anti-apoptotic pathways (e.g., P-3 kinase and STATS), leading to the death of the Bcr-Abl phenotype cells and thereby providing an effective therapy against CML. The combinations of the present invention useful as Bcr-Abl inhibitors are thus especially appropriate for the therapy of diseases related to its overexpression, especially leukemias, such as leukemias, e.g., CML or ALL.

The RAF kinase inhibiting property of the combinations of the present invention makes them useful as therapeutic agents for the treatment for proliferative diseases characterized by an aberrant MAP kinase signaling pathway, particularly many cancers characterized by overexpression of RAF kinase or an activating mutation of RAF kinase, such as melanoma having mutated B-RAF, especially wherein the mutated B-RAF is the V599E mutant. The present invention also provides a method of treating other conditions characterized by an aberrant MAP kinase signaling pathway, particularly where B-RAF is mutated, e.g., benign Nevi moles having mutated B-RAF, with the combinations of the present invention.

In general, the disease characterized by excessive signaling through the MAP kinase signaling pathway is a proliferative disease, particularly a cancer characterized by increased RAF kinase activity, e.g., one which overexpresses wild-type B- or C-RAF kinase, or that expresses an activating mutant RAF kinase, e.g., a mutant B-RAF kinase. Cancers wherein a mutated RAF kinase has been detected include melanoma, colorectal cancer, ovarian cancer, gliomas, adenocarcinomas, sarcomas, breast cancer and liver cancer. Mutated B-RAF kinase is especially prevalent in many melanomas.

In accordance with the present invention, a sample of diseased tissue is taken from the patient, e.g., as a result of a biopsy or resection, and tested to determine whether the tissue produces a mutant RAF kinase, such as a mutant B-RAF kinase or overexpresses a wild-type RAF kinase, such as wild-type B- or C-RAF kinase. If the test indicates that mutant RAF kinase is produced or that a RAF kinase is overproduced in the diseased tissue, the patient is treated by administration of an effective RAF-inhibiting amount of a RAF inhibitor compound described herein.

Further in accordance with the invention is the use of combinations of the present invention described herein for the preparation of a medicament for the treatment of melanoma which comprises: (a) testing melanoma tissue from the patient to determine whether the melanoma tissue expresses mutant RAF kinase or overexpresses a wild-type RAF kinase; and (b) treating the patient if the melanoma tissue is found to overexpress a wild-type RAF kinase or express an activating mutant B-RAF kinase with an effective RAF kinase inhibiting amount of combinations of the present invention.

However, it is also possible to down-regulate the MAP kinase signaling pathway with a RAF kinase inhibiting compound If another kinase in the cascade is the cause of excessive signaling in the pathway. Thus, the present invention further relates to the treatment of a disease characterized by excessive signaling in the MAP kinase signaling pathway attributed to a cause other than an activating mutation in or overexpression of a RAF kinase.

The combinations of the present invention primarily inhibit the growth of blood vessels and are thus, e.g., effective against a number of diseases associated with deregulated angiogenesis, especially diseases caused by ocular neovascularisation, especially retinopathies, such as diabetic retinopathy or age-related macula degeneration, psoriasis, haemangioblastoma, such as haemangioma, mesangial cell proliferative disorders, such as chronic or acute renal diseases, e.g., diabetic nephropathy, malignant nephrosclerosis, thrombotic microangiopathy syndromes or transplant rejection, or especially inflammatory renal disease, such as glomerulonephritis, especially mesangioproliferative glomerulonephritis, haemolytic-uraemic syndrome, diabetic nephropathy, hypertensive nephrosclerosis, atheroma, arterial restenosis, autoimmune diseases, diabetes, endometriosis, chronic asthma, and especially neoplastic diseases (solid tumors, but also leukemias and other haematological malignancies), such as especially breast cancer, cancer of the colon, lung cancer (especially small-cell lung cancer), cancer of the prostate or Kaposi's sarcoma. Combinations of the present invention inhibit the growth of tumours and are especially suited to preventing the metastatic spread of tumors and the growth of micrometastases.

The inhibition of endogenous FAK signalling results in reduced motility and in some cases induces cell death. On the other hand, enhancing FAK signalling by exogenous expression increases cell motility and transmitting a cell survival signal from ECM. In addition, FAK is overexpressed in invasive and metastatic epithelial, mesenchymal, thyroid and prostate cancers. Consequently, an inhibitor of FAK is likely to be a drug for anti-tumor growth and metastasis. The compounds are thus indicated, e.g., to prevent and/or treat a vertebrate and more particularly a mammal, affected by a neoplastic disease, in particular, breast tumor, cancer of the bowel (colon and rectum), stomach cancer and cancer of the ovary and prostate, non-small cell lung cancer, small cell lung cancer, cancer of liver, melanoma, bladder tumor and cancer of head and neck.

The invention relates to a method of treating myeloma, especially myeloma which is resistant to conventional chemotherapy. The term “myeloma”, as used herein, relates to a tumour composed of cells of the type normally found in the bone marrow. The term “multiple myeloma”, as used herein, means a disseminated malignant neoplasm of plasma cells which is characterized by multiple bone marrow tumor foci and secretion of an M component (a monoclonal immunoglobulin fragment), associated with widespread osteolytic lesions resulting in bone pain, pathologic fractures, hypercalcaemia and normochromic normocytic anaemia. Multiple myeloma is incurable by the use of conventional and high-dose chemotherapies. The invention relates to a method of treating myeloma, especially myeloma which is resistant to conventional chemotherapy.

A preferred embodiment of the present invention is the combination of a RAF inhibitor and a JAK kinase inhibitor for the treatment of myelomas, especially multiple myeloma. Most especially preferred is the combination of a RAF inhibitor selected from:
(4-tert-butyl-phenyl)-(4-pyridin-4-ylmethyl-isoquinolin-1-yl)-amine;
[4,7′]biisoquinolinyl-1-yl-4-(tert-butyl-phenyl)-amine;
(4-tert-butyl-phenyl)-(4-quinazolin-6-yl-isoquinolin-1-yl)-amine;
[4,7′]biisoquinolinyl-1-yl-(2-tert-butyl-pyrimidin-5-yl)-amine;
   and combinations thereof, and a JAK kinase inhibitors selected from:
N-benzyl-3,4-dihydroxy-benzylidene-cyanoacetamide α-cyano-(3,4-dihydroxy)-]N-benzylcinnamamide (Tyrphostin AG 490); prodigiosin 25-C (PNU156804);
[4-(4′-hydroxyphenyl)-amino-6,7-dimethoxyquinazoline] (WHI-P131);
[4-(3′-bromo-4′-hydroxylphenyl)-amino-6,7-dimethoxyquinazoline] (WHI-P154);
[4-(3′,5′-dibromo-4′-hydroxylphenyl)-amino-6,7-dimethoxyquinazoline] WHI-P97; and
3-{(3R,4R)-4-methyl-3-[methyl-(7H-pyrrolo[2,3-d]pyrimidin-4-yl)-amino]-piperidin-1-yl}-3-oxo-propionitrile,
   in free form or in salt form, e.g., mono-citrate (also called CP-690,550) for the treatment of myeloma, especially multiple myeloma.

We claim:

1. A pharmaceutical combination comprising:
a) at least one agent selected from Bcr-Abl, Flt-3, FAK and RAF kinase inhibitors; and
b) at least one subtype-selective or subtype unselective JAK kinase inhibitor.

2. A method for treating or preventing a proliferative disease in a subject in need thereof, comprising co-administration to said subject, e.g., concomitantly or in sequence, of a therapeutically effective amount of at least one agent selected from Bcr-Abl, Flt-3, FAK and RAF kinase inhibitors and at least one JAK3 kinase inhibitor.

3. A pharmaceutical combination according to claim 1, for use in a method according to claim 2.

4. A pharmaceutical combination according to claim 1, for use in the preparation of a medicament for use in a method according to claim 2.

5. A pharmaceutical combination according to claim 1, wherein agent a) is selected from:
N-{5-[4-(4-methyl-piperazino-methyl)-benzoylamido]-2-methyl-phenyl}-4-(3-pyridyl)-2-pyrimidine-amine;
4-methyl-3-[[4-(3-pyridinyl)-2-pyrimidinyl]amino]-[5-(4-methyl-1H-imidazol-1-yl)-3-(trifluoromethyl)phenyl]benzamide;
N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide;
1-[4-(4-ethyl-piperazinyl-1-ylmethyl)-3-trifluoromethyl-phenyl]-3-[4-(6-methylamino-pyrimidin-4-yloxy-phenyl]-urea;
1-[4-(2-amino-pyrimidin-4-yloxy)-phenyl]-3-[4-(4-ethyl-piperazin-1-ylmethyl)-3-trifluoromethyl-phenyl]-urea;
1-[4-(2-amino-pyrimidin-4-yloxy)-phenyl]-3-[4-(4-methyl-piperazin-1-ylmethyl)-3-trifluoromethyl-phenyl]-urea;
(4-tert-butyl-phenyl)-(4-pyridin-4-ylmethyl-isoquinolin-1-yl)-amine;
[4,7′]biisoquinolinyl-1-yl-4-(tert-butyl-phenyl)-amine;
(4-tert-butyl-phenyl)-(4-quinazolin-6-yl-isoquinolin-1-yl)-amine;
[4,7′]biisoquinolinyl-1-yl-(2-tert-butyl-pyrimidin-5-yl)-amine;
2-[5-chloro-2-(2-methoxy-4-morpholin-4-yl-phenylamino)-pyrimidin-4-ylamino]-N-methyl-benzamide; and
N3-[4-(4-morpholin-4-yl-cyclohexyl)-phenyl]-1-pyridin-2-yl-1H-[1,2,4]triazole-3,5-diamine;
and combinations thereof.

6. A pharmaceutical combination according to claim 1, wherein agent b) is selected from:
N-benzyl-3,4-dihydroxy-benzylidene-cyanoacetamide α-cyano-(3,4-dihydroxy)-]N-benzylcinnamamide (Tyrphostin AG 490); prodigiosin 25-C (PNU156804);
[4-(4′-hydroxyphenyl)-amino-6,7-dimethoxyquinazoline] (WHI-P131);
[4-(3′-bromo-4′-hydroxylphenyl)-amino-6,7-dimethoxyquinazoline] (WHI-P154);
[4-(3′,5′-dibromo-4′-hydroxylphenyl)-amino-6,7-dimethoxyquinazoline] WHI-P97; and
3-{(3R,4R)-4-methyl-3-[methyl-(7H-pyrrolo[2,3-d]pyrimidin-4-yl)-amino]-piperidin-1-yl}-3-oxo-propionitrile,
in free form or in salt form, e.g., mono-citrate (also called CP-690,550); and combinations thereof.

7. A method according to claim 2, wherein agent a) is selected from:
N-{5-[4-(4-methyl-piperazino-methyl)-benzoylamido]-2-methyl-phenyl}-4-(3-pyridyl)-2-pyrimidine-amine;
4-methyl-3-[[4-(3-pyridinyl)-2-pyrimidinyl]amino]-N-[5-(4-methyl-1H-imidazol-1-yl)-3-(trifluoromethyl)phenyl]benzamide;
N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide;
1-[4-(4-ethyl-piperazinyl-1-ylmethyl)-3-trifluoromethyl-phenyl]-3-[4-(6-methylamino-pyrimidin-4-yloxy-phenyl]-urea;
1-[4-(2-amino-pyrimidin-4-yloxy)-phenyl]-3-[4-(4-ethyl-piperazin-1-ylmethyl)-3-trifluoromethyl-phenyl]-urea;
1-[4-(2-amino-pyrimidin-4-yloxy)-phenyl]-3-[4-(4-methyl-piperazin-1-ylmethyl)-3-trifluoromethyl-phenyl]-urea;
(4-tert-butyl-phenyl)-(4-pyridin-4-ylmethyl-isoquinolin-1-yl)-amine;
[4,7′]biisoquinolinyl-1-yl-4-(tert-butyl-phenyl)-amine;
(4-tert-butyl-phenyl)-(4-quinazolin-6-yl-isoquinolin-1-yl)-amine; and
[4,7′]biisoquinolinyl-1-yl-(2-tert-butyl-pyrimidin-5-yl)-amine;
2-[5-chloro-2-(2-methoxy-4-morpholin-4-yl-phenylamino)-pyrimidin-4-ylamino]-N-methyl-benzamide; and
N3-[4-(4-morpholin-4-yl-cyclohexyl)-phenyl]-1-pyridin-2-yl-1H-[1,2,4]triazole-3,5-diamine;
and combinations thereof.

8. A method according to claim 2, wherein agent b) is selected from:
N-benzyl-3,4-dihydroxy-benzylidene-cyanoacetamide α-cyano-(3,4-dihydroxy)-]N-benzylcinnamamide (Tyrphostin AG 490); prodigiosin 25-C (PNU156804);
[4-(4′-hydroxyphenyl)-amino-6,7-dimethoxyquinazoline] (WHI-P131);
[4-(3′-bromo-4′-hydroxylphenyl)-amino-6,7-dimethoxyquinazoline] (WHI-P154);
[4-(3′,5′-dibromo-4′-hydroxylphenyl)-amino-6,7-dimethoxyquinazoline] WHI-P97;
3-{(3R,4R)-4-methyl-3-[methyl-(7H-pyrrolo[2,3-d]pyrimidin-4-yl)-amino]-piperidin-1-yl}-3-oxo-propionitrile,
in free form or in salt form, e.g., mono-citrate (also called CP-690,550); and combinations thereof.

9. A method for myeloma comprising administering a combination of a RAF inhibitor and a JAK kinase inhibitor wherein the RAF inhibitor is selected from:
(4-tert-butyl-phenyl)-(4-pyridin-4-ylmethyl-isoquinolin-1-yl)-amine;
[4,7′]biisoquinolinyl-1-yl-4-(tert-butyl-phenyl)-amine;
(4-tert-butyl-phenyl)-(4-quinazolin-6-yl-isoquinolin-1-yl)-amine;
[4,7′]biisoquinolinyl-1-yl-(2-tert-butyl-pyrimidin-5-yl)-amine;
and combinations thereof, and the JAK kinase inhibitor is selected from:
N-benzyl-3,4-dihydroxy-benzylidene-cyanoacetamide α-cyano-(3,4-dihydroxy)-]N-benzylcinnamamide (Tyrphostin AG 490); prodigiosin 25-C (PNU156804);
[4-(4′-hydroxyphenyl)-amino-6,7-dimethoxyquinazoline] (WHI-P131);
[4-(3′-bromo-4′-hydroxylphenyl)-amino-6,7-dimethoxyquinazoline] (WHI-P154);
[4-(3′,5′-dibromo-4′-hydroxylphenyl)-amino-6,7-dimethoxyquinazoline] WHI-P97;
3-{(3R,4R)-4-methyl-3-[methyl-(7H-pyrrolo[2,3-d]pyrimidin-4-yl)-amino]-piperidin-1-yl}-3-oxo-propionitrile,
in free form or in salt form, e.g., mono-citrate (also called CP-690,550); and combinations thereof.
